(19) United States
(12) Patent Application Publication
(10) Pub. No.: US 2008/0075772 A1
(43) Pub. Date: Mar. 27, 2008

(54) PHARMACEUTICAL COMPOSITIONS HAVING NOVEL SCORING PATTERNS AND METHODS OF USING THOSE COMPOSITIONS

(76) Inventors: Lawrence Solomon, Boca Raton, FL (US); Allan S. Kaplan, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 11/735,268

(22) Filed: Apr. 13, 2007

Related U.S. Application Data
(60) Provisional application No. 60/791,834, filed on Apr. 13, 2006.
(60) Provisional application No. 60/792,601, filed on Apr. 17, 2006.
(60) Provisional application No. 60/792,933, filed on Apr. 18, 2006.
(60) Provisional application No. 60/861,898, filed on Nov. 30, 2006.

(51) Int. Cl. A61K 31/56; A61K 31/5513; A61K 31/515; A61K 31/401; A61K 31/366; A61K 31/22; A61K 31/203; A61K 9/20; A61K 9/44

(52) U.S. Cl. 424/464; 424/467; 514/221; 514/559; 514/594; 514/270; 514/725; 514/169; 514/423; 514/548; 514/460

(57) ABSTRACT

The invention provides novel scoring patterns for a wide variety of tablets that contain specific drugs or contain drugs in certain drug classes, and methods of treatment with layered tablets and fractional tablets derived from breaking said tablets.

### CROSS-REFERENCE TO RELATED APPLICATIONS

This is a continuation-in-part of U.S. Provisional Application Ser. No. 60/792,601 filed Apr. 17, 2006; and is also a continuation-in-part of U.S. Provisional Application Ser. No. 60/792,933 filed Apr. 18, 2006; and is also a continuation-in-part of U.S. Provisional Application Ser. No. 60/861,898 filed Nov. 30, 2006.

### BACKGROUND OF THE INVENTION

It is known to create a pharmaceutical tablet with one or more indentation, mark, or score line (hereinafter, “score”). Typically the score is a bisecting score, which may provide a suggestion that the scored tablet is divisible into two portions. Other patterns such as trisection, including trisection in the presence of bisection, which may suggest divisibility into at least three portions, and quadrisection, which may suggest divisibility into four sections, are known but are far rarer than bisection. Even scored tablets, however, may be labeled to instruct the user to swallow the tablet whole, and therefore contraindicate any suggestion of divisibility for the tablet. Accordingly, a score may be provided in a tablet for purposes other than breakability. Major uses of scores include providing structural strength to the tablet and decorative functions. Herein, a “functional” score is one that is designed to provide breakability functionality to the tablet.

Although scores are known to be provided in pharmaceutical tablets, it is well documented that breaking of scored tablets provides potentially significant variation in the precision of any resultant dose in the resultant tablet portion. In general, tablet portions (hereinafter, “tablettes”) resulting from currently manufactured scored tablets provide doses of less accuracy than are produced by separately creating a smaller (whole) dose, as in a separately manufactured dosage form containing that lower dose. Moreover, for tablets having a scoring pattern such as trisection or quadrisection, which are more complex that the bisecting score pattern, one observation is that when a tablet is broken and then a smaller part (tablette) is broken again, such as typically occurs when breaking a tablet into three or four parts in two or more breaking steps, any imprecision in the predicted dose in a portion resulting from the first breaking carries over and is additive to any such imprecision in a subsequent breaking of that portion.

As used herein, the term “quadrisected” (or, “quadrisect, ” “quadrisection,” etc.) refers to a crossing scoring pattern or three parallel score marks, so that breaking the tablet precisely as guided by said two or three scores would in theory provide four equal tablettes, each containing one-fourth the mass/drug quantity as the whole tablet. This is differentiated from the scoring pattern utilized in the Dividose® technology (Desyrel® (trazodone) and Buspar® (buspirone) in which two parallel scores on one surface of the tablet trisect the tablet, and a bisecting score is positioned on a different, contiguous surface.

Known quadrisected tablets include without limitation:
Alprazolam (anti-anxiety)
Captopril, enalapril, lisinopril (ACE inhibitors)
Furosemide (diuretic)
Atropine, scopolamine (anti-cholinergics)
Chlorpropamide, glicazide (sulfonylurca hypoglycemics)
Methadone (narcotic)
Methylprednisolone (corticosteroid)
Simethicone (anti-flatulent)
Paroxetine (antidepressant)
Carbamazepine (anti-neuropathic agent, anti-convulsant)
Tizanidine (antispasmodic)
Paracetacod (narcotic)—combination product containing codeine and ascorbic acid

Known bisected/trisected tablets include:
Buspirone (Buspar Dividose®)
Trazodone (Desyrel Dividose®)

Known trisected tablets that are not also bisected on a separate surface: None

In view of the large number of tablet or tablettable products that have a 1:2:3 or a 1:2:4 milligram ratio for marketed strengths of manufactured dosage forms or therapeutic doses, certain benefits can be achieved by providing tablets having a unique scoring pattern that enables portions or sections of tablets broken along those unique scores to predictably and accurately be split into dosages of one-quarter, one-third, half, two-thirds, or three-quarters of the whole tablet.

On a product-by-product basis, reasons for not providing scoring patterns more complex than bisected scoring patterns have included the desire for accurate subdividing of doses, where potential magnification of error by multiple subdivisions apparently existed. For example, warfarin sodium is an anticoagulant provided as a bisected tablet. A study published in the Journal of the American Medical Association (JAMA) of elderly patients using tablet splitters found major variations were present in the sub-dose portions resulting from “halving” this scored tablet. Given the importance of accurate dosing with warfarin, it has, in the past, been important that quadrisection not be performed with manufactured warfarin product, lest it induce patients, aides, and other users to break the tablet into four pieces, as further “halving” of the two inaccurately divided “halves,” (i.e., “quartering” of the whole tablet) can magnify the error caused by the initial “halving” of the tablet. It has not been taught or suggested to produce a quadrisected warfarin tablet.

In another example, it is known that anticonvulsants frequently undergo dose adjustment, and that accurate dosing is important. The well known anticonvulsant Lamictal® (lamotrigine) is provided as scored tablets in the U.S. but per the Product Information (“PI” or “label”) for Lamictal, the tablets are to be taken whole and not broken. It has therefore not been taught or suggested to provide a quadrisect pattern on a Lamictal tablet. This product provides an excellent example that the presence of a score on a tablet does not suggest that the tablet is made to be broken. Thus a scored tablet cannot be assumed to be intended to be used in a dosage adjustment scheme.

In many other cases, such as anti-psychotics and various drugs for such as psychiatric, neurologic, or cardiovascular uses, trisection or quadrisection has, within a class, either never been done; or has been done rarely, and generally without a teaching or suggestion to break those tablets into portions according to the scoring pattern provided on those tablets.

Along with a pharmaceutical manufacturer's avoidance of inappropriate dosing by inducing or implicitly or explicitly recommending breaking a tablet one or more times, another reason that certain tablets have not been provided with a score more complex than a bisecting score may involve the physical characteristics or properties of the tablets. For example, hardness and other breaking characteristics of a particular drug formulation available in the prior art may contribute to the unsuitability of such complex scoring, or perhaps even for bisecting.

The importance of improved pharmaceutical tablets and of improved and novel methods of treatment that preferably include tablets that break into “tablettes” of predictable dose is seen by a proprietary survey performed by ACCU-BREAK Pharmaceuticals, Inc. in 2005. Six out of six practicing physicians agreed that accurate breaking of tablets was medically important for a number of drug classes, such as ACE inhibitors, angiotensin receptor blockers, oral hypoglycemics, etc. The pharmaceutical industry has paid insufficient attention to this medically important topic. The invention is aimed at improving the practice of medicine and pharmaceutics with novel drug products involving novel scoring patterns as well as novel methods of treatment, which may or may not utilize drug products that are scored in a novel pattern.

Certain tablets may not be scored and may be especially inappropriate for complex scoring beyond bisecting, because of a need to be film-coated. Certain preferred compositions of the subject invention can provide a benefit for permitting unique scoring patterns on or in tablets that may be coated.

Recent practice in the pharmaceutical industry has apparently been away from quadrisection of pharmaceutical tablets, though one relatively recent introduction of a quadrisected tablet was a paroxetine product in South Africa (marketed under the brand name, “Parax”), for which the 40 mg tablet is quadrisected, according to its label.

To the inventor's knowledge, drug product information (e.g., drug product labeling) has not before indicated that a broken portion of a scored tablet could be used in place of or as an equivalent of a separately produced dosage form of the strength implied by breaking the whole product in accordance with the scoring pattern. A recent European Pharmacopoeia (“EP”) requirement that scored tablets must be breakable into predictable strengths within tight specifications has recently been relaxed, apparently due to the difficulty the pharmaceutical industry had in meeting its strictures. It has therefore been previously unachievable to propound a method of dose titration (up or down) that involves utilizing a predictable, accurate partial (e.g., half, third, or quarter) dose by breaking an unscored or scored dosage form by breaking a whole dosage form to provide more than one portion, and then further breaking of one of those portions.

Although patients and other persons have broken some scored and unscored tablets to provide lower doses, those previous methods of breaking tablets could not assure a patient was reliably ingesting a predictable, accurate lower dose, such as a 20 mg dose from a halved 40 mg tablet. Rather, previous compositions and methods could only provide two doses that are each less than the dose of the whole tablet (taking note of the loss of mass due to crumbling or chipping on tablet breaking).

Recently, Solomon and Kaplan have disclosed novel dosage forms that comprise a layered tablet structure containing a preferably pharmacologically inactive segment that serves as a preferred breaking region if tablet subdivision is desired. See, for example, WO 2005/112898 and WO 2005/112900, which are incorporated herein by reference in their entirety.

It is now recognized that the subject compositions and methods of treatment may be employed by using dosage forms that can be broken to provide predictably accurate lower doses, and preferably employing layered dosage forms as described herein. These compositions and methods can be advantageously and beneficially incorporated into medical, nursing, or institutional treatment plans, as well as in PI's for drug products.

### SUMMARY OF THE INVENTION

The invention in certain embodiments provides compositions, e.g., pharmaceutical tablets, comprising novel scoring patterns formed in those compositions. The novel scoring patterns can be used in pharmaceutical products spanning a wide variety of drug classes and specific drug products within those classes. An object of the invention is to allow enhanced dosage flexibility (i.e., using a given strength of a tablet to yield a smaller and preferably predictable, accurate strength) over the current art, in a manner that benefits the practice of medicine and pharmaceutics. Making use of advances in dosing flexibility, the invention involves all pharmaceutical products such as tablets or capsules that have not been made in a bisected, trisected or quadrisected (or pentasected, etc.) tablet form.

The invention preferably relates to compressed tablets but is not limited to such a mode of manufacture of pharmaceutical tablets.

The invention concerns pharmaceutical tablets for which more than one useful dose is known, especially where three useful doses representing a ⅓, ⅔, and whole dose are known (for trisected tablets of the invention), where a ¼, ½, and whole dose are known (for quadrisected tablets of the invention), and the like. In the case of breaking a tablet into two or more tablettes, for example, the subject invention provides divided doses that can advantageously meet FDA regulations of individually made tablets of the lower strength. For example, two tablettes formed from a 40 mg tablet can provide a predictable 20 mg dose in each tablet half, where those tablet halves can meet FDA requirements for an individually manufactures 20 mg dose.

There is thus no limitation to the classes or specific names of drugs that have been produced as pharmaceutical products. The examples described and claimed herein are exemplary only. Compositions and the methods of using those compositions in accordance with the subject invention include any active pharmaceutical ingredient, including but not limited to the active ingredients listed in the FDA publication Approved Drug Products with Therapeutic Equivalence Evaluations, 27^{th} Edition, and its supplements. This publication is commonly known as the “Orange Book,” the relevant portions of which are hereby incorporated by reference.

Except for the specific examples of trisected and quadrisected tablets listed above, the drug classes that involve solid oral dosage forms, and the specific drug products that involve solid oral dosage forms, are claimed for the subject invention.

Among the preferred embodiments of the invention are tablets that can provide predictable, accurate doses when broken into tablet portions, known as “tablettes.” For example, tablets comprising unitary segments and an inactive substrate or base layer, as described by Solomon and Kaplan in WO 2005/112900 and WO 2006/038916, or layered or segmented tablets as described in WO 2005/112870, WO 2005/112897, and WO 2005/112898. Accurately breakable tablets are also described in U.S. Published Application No. 2002/0052411, U.S. Pat. Nos. 6,488,939, 6,692,765, 6,342,248, 5,756,124, 5,520,929, 4,258,027, 3,723,614, 3,336,200, Re. 29077, and UK Patent GB 2351234A, though certain of the tablet designs described therein may not be suitable for other than bisected tablets. The disclosures of each of these published PCT applications, and the issued U.S. Patents, above, incorporated herein by reference.

Among the tablets more preferred for the invention are, without limitation, drug products of the following classes: Anticoagulants, antihypertensives, antipsychotics, tranquilizers, anticonvulsants, hypoglycemics, antidepressants, and steroids such as corticosteroids.

Many members of the above classes of drug products may frequently undergo dosage adjustment on initiation and discontinuance of, and during drug treatment.

The subject invention includes, without limitation, pharmaceutical tablets comprising at least one “functional” score wherein such pharmaceutical products have heretofore never included a functional score, even if the score pattern is superficially similar to that known for a product comprising a particular drug. This category includes without limitation a functional bisecting score in a product previously only containing a non-functional bisecting score, a functional trisecting score in a product previously only containing a non-functional trisecting score, a functional quadrisecting score in a product previously only containing a non-functional quadrisecting score, and the like. These functional scores are defined as scores that are dimensioned such that by breaking through the score, a smaller tablet (i.e., a tablette) or tablets is (are) provided that contains a substantially accurate or predictable partial dose. Examples of non-functional scores are given to include, without limitation, decorative scores and scores that aid the structural stability of the tablet. The latter is said to be the reason that scoring of tablets was in fact introduced.

Another aspect of the invention is to utilize drug products that are manufactured in a dosing scheme that would lend itself to bisection or quadrisection, such as with four doses that are doubles of each other, such as 5, 10, 20, and 40 mg, tablets, but instead produce such products in a trisection fashion, either involving trisection of a known dosage strength such as a 40 mg tablet or a new dosage strength such as 22.5.

In a similar spirit to the above, certain drug products such as warfarin are produced for the U.S. market in dose strengths that are doubles, trebles, and quadruples of lower manufactured doses (e.g. 1, 2, and 4 mg tablets; and, 22.5, and 7.5 mg tablets). The invention provides, as examples, for a trisected dosage strength that is not a treble dose of a lower manufactured dose, or for quadrisected dosage strength of a dose that is not a quadruple dose of a lower manufacture dose; or, the invention provides for trisection or quadrisection of known tablet strengths.

Also within the spirit and metes and bounds of the invention is a bisecting score of a drug product wherein the drug product has only one approved dose, such as Zetia® (10 mg), where it has not been taught or suggested to provide a bisected 10 mg or 20 mg tablet, or a drug product such as Proscar® (finasteride 5 mg), which has a different indication for the 1 mg finasteride product, marketed under the brand name Propecia®. In this latter instance, the subject invention can provide a quadrisected 5 mg tablet which when broken can yield a 2.5 or 1.25 mg product, or a “pentasected” product (e.g., a tablet with four parallel scores) which when broken into its indicated portions through the scores can yield five 1-mg products. Thus, yet another aspect of the invention relates to a score that guides division of a tablet into fifths, referred to herein as “pentasected.”

One embodiment of the subject invention includes a method of breaking tablets that predictably or reliably create accurate lower doses in the resultant broken tablet portions. It is a primary object of the invention to provide a method of treating patients to a numerical goal such as a desired blood pressure, cholesterol level, or thyroid-stimulating hormone level, or to reach a desired clinical or therapeutic endpoint, such as reduction in anxiety, depression, or asthma, or seizures. The method of the invention can be utilized in the treatment of chronic conditions such as hypertension and hyperlipidemia. The invention in certain embodiments involves the use of finished dosage forms, e.g., tablets, by breaking or otherwise dividing them to produce a predictably accurate smaller or lower dose (or sub-dose, also referred to herein as a fractional dose). This advantageously allows tablet breaking to be recommended and even preferred in treatment plans, authoritative guidelines, manufacturer's product information, and the like.

Certain preferred embodiments of the invention also involve written instructions and use of the subject compositions and methods for upward dose titration, such as are commonly used for medical treatment of disorders involving cholesterol, diabetes mellitus, hypertension, thyroid disorders, and epilepsy, and are often utilized near the initiation of treatment with the drug product of a subject composition. Also within the scope of the invention are downward dose titration regiments that for example may be used with treatment of acute allergic or asthmatic reaction using prednisone, in anti-epileptic treatments with a second medication while decreasing the dose of a first medication, or in the withdrawal of certain antihypertensive products such as clonidine or a beta blocker.

A further embodiment of the invention involves treatment plans and other usage that utilize alternating doses or doses that are in between the doses of available whole dosage forms. For example, for a product such as Lamictal that is only available in the U.S. in 25 and 100 mg strength tablets, the compositions and methods of the subject invention can provide for a 37.5 mg dose by allowing accurate and predictable breaking of a 25 mg tablet into two tablettes, each containing 12.5 mg, then administering one 25 mg tablette (1½ of the 25 mg tablets). Advantageously, the subject invention in certain preferred embodiments can help assure that administering three 12.5 mg tablettes (each formed from the breaking of a 25 mg tablet) or three separately made 12.5 mg tablets will be equivalent in total administered dose to the above-described dosing using one 25 mg tablet plus one 12.5 mg tablette.

A related embodiment of the invention involves clinical situations in which, for example, a nurse in a hospital has received and intends to administer to a patient a unit dose of a 20 mg tablet containing a drug, e.g., lisinopril, but a treating physician writes an order to lower the dose to 10 mg prior to its administration. The nurse may comply with the order by, according to the subject invention, breaking a 20 mg tablet that provides an accurate, predictable 10 mg dose that is substantially equivalent to an individually manufactured 10 mg lisinopril tablet. Prior to the invention, it would not have been feasible to provide a half tablet with the reasonable assurance that said half tablet would be substantially equivalent to an individually manufactured 10 mg lisinopril tablet, especially given the uncertainty of who is breaking the 20 mg tablet and under what conditions.

The invention also involves novel methods of dose adjustment by breaking whole tablets or tablettes already formed by breaking a larger tablet structure such as a whole tablet, thus utilizing fractional tablets (tablettes) for dosing. These methods often include the ability of a patient who may be elderly, frail, legally blind, etc. to perform the dosage adjustment in a medically and pharmaceutically acceptable manner.

The invention also involves novel methods of dose adjustment utilizing tablets where such has not been taught or suggested, even if the tablet cannot be broken with predictable accuracy by the intended user, or where the tablet cannot be broken with predictable accuracy by a pharmacist or expert tablet splitter. These embodiments of the invention involve numerous drug products within numerous classes of drug products. While less preferred than tablets that readily break to provide a predictable dose, they represent improvements in the art in their novelty and in the medical benefits they provide. Non-limiting examples of such methods are described subsequently herein.

Thus, the embodiments of the subject invention include an improved pharmaceutically acceptable tablet containing at least one drug wherein said at least one drug is provided in a prior art dosage form as a capsule, an unscored tablet, a tablet having a bisecting score pattern, a tablet having a trisecting score pattern, or a tablet having a quadrisecting score pattern, said improvement comprising:
a tablet when the prior art dosage form is not a tablet or has not been tabletted;
a tablet having a score pattern selected from the group consisting of a bisecting, trisecting, quadrisecting, a pentasecting, and a score pattern more complex than a pentasecting score pattern, when the prior art tablet is limited to an unscored tablet;
a tablet having a score pattern selected from the group consisting of a trisecting, quadrisecting, a pentasecting, and score pattern more complex than a pentasecting score pattern when the prior art tablet is limited to an unscored tablet or a bisected tablet;
a tablet having a score pattern selected from the group consisting of a bisecting, quadrisecting, a pentasecting, and a score pattern more complex than a pentasecting score pattern, when the prior art dosage form is a trisected tablet; or
a tablet having a score pattern selected from the group consisting of a bisecting, trisecting, and a pentasecting score pattern when the prior art dosage form is a quadrisected tablet; wherein said score pattern is formed by a debossed score or separation mark.

The above tablets can comprise a novel scoring pattern heretofore not used in any dosage form incorporating the specific drug or drugs. These scoring patterns can be conventional bisecting scoring patterns or can define five or more substantially equal regions in which a substantially equal amount of drug or combination of drugs is present in each regions defined by the score pattern. Alternatively, the tablet can be readily divisible into equal amounts of drug or drugs. Preferably, the tablets of the subject invention are layered or segmented tablets and can include active and inactive layers or segments within a single tablet. Further preferred embodiments are tablets that are readily breakable, e.g., manually breakable without requiring a commercially available tablet splitter or other cutting device, into predictable fractional doses or dosings.

The tablets of the subject invention can comprise any pharmaceutically acceptable active pharmaceutical ingredient, and preferably one from the group of: an anticoagulant, a statin, a non-statin lipid modifier, a cardiovascular agent, an antihypertensive agent, an angiotensin receptor antagonist, an antipsychotic agent, an atypical antipsychotic agent, a hypnotic agent, a benzodiazepine, and antidepressant, a steroid, a hypoglycemic agent, an anticonvulsant, a GABA (gamma-aminobutyric acid) receptor agonists, an antihistaminergic compound, a glitazone, a retinoid, a direct rennin inhibitor, an anti-retroviral agent, an immunosuppressant, a proton pump inhibitor, a barbiturate, and a narcotic. A salt, isomer, polymorph, hydrate, metabolite, prodrug, or derivative of the active pharmaceutical ingredient is considered and included as part of the invention.

Another embodiment of the subject invention concerns a method of treating, preventing or ameliorating a medical condition in a human or other animal using a dosing regimen comprising administration of different doses the human or other animal, the method comprising:
providing a whole tablet comprising a dose of one or more drugs, said tablet being breakable to provide at least two portions of said tablet such that each portion comprises a predictable fractional dose of said one or more active drugs contained in the whole tablet, said fractional dose being more predictable than a fractional dose available from a prior art dosage form manufactured using known formulation technologies and comprising the same drug or drugs;
prescribing or administering a fractional dose of said one or more active drugs, said fractional dose obtained by breaking the whole tablet into its one or more portions comprising a predictable fractional dose.

A preferred embodiment of the subject method is where the fractional dose is a predictable half, third, or quarter fractional dose of the dose contained in the whole tablet. More preferably, the method of accurate braking of the tablet or a portion thereof is readily performed by a patient in a home setting and can be readily performed by a elderly, arthritic, or blind patient. Still more preferable is performing the method using a tablet breaking into predictable fractional doses, such as a layered or segmented tablet. The subject method can preferably be used in treating, ameliorating, or preventing such conditions as hypertension, hyperlipidemia, diabetes mellitus, hypothyroidism, benign prostatic hyperplasia, asthma, pain, and allergy.

It would be understood by persons of ordinary skill in the art, in light of the subject disclosure, that the subject method can be useful in administering an initial dose in an increasing dose titration regimen, integral with a decreasing dose titration regimen, or in a dosing regimen where the dose may need to be adjusted up or down according to a patient response. Advantageously, the subject method, employing the administration of the fractional dose, can facilitate reaching a desired therapeutic or clinical end-point in treating said medical condition for the patient.

The subject invention further includes an article of manufacture or kit. The article of manufacture or kit can include:
a whole dosage form comprising a medicament, said dosage form being accurately and predictably breakable into a plurality of fractional doses of a drug or drugs, and a separate instruction to create the fractional dose by breaking said whole dosage form or a fractional dose thereof into the plurality of fractional doses of said drug or drugs, wherein said dosage form and instruction are co-packaged as a single unit. Preferably, the instruction also includes notification that breaking the tablet as directed will produce a predictable dose of said drug or drugs.

The subject invention further includes a method of producing a predictable dose of a drug or drugs contained in a whole tablet, said method comprising breaking a tablet of the invention into a plurality of tablettes and administering a tablette to provide a fraction of the whole tablet's dose of the drug or drugs. This method is preferably used in a dosing regimen comprising administration of a plurality of doses.

Compositions of the invention are assumed herein to be pharmaceutically acceptable. The term “tablet” herein is used to mean a pharmaceutical tablet.

### BRIEF DESCRIPTION OF THE DRAWINGS

FIGS. 1A-1C depict embodiments of cross-scored warfarin tablets according to the subject invention: FIG. 1A shows a trisected tablet breakable into three portions; FIG. 1B shows a quadrisected tablet breakable into four portions; and FIG. 1C shows a pentasected tablet breakable into five portions.

FIGS. 2A-2C depict embodiments of warfarin tablets according to the subject invention comprising a plurality of parallel scores provided on the same surface of the tablet: FIG. 2A shows a trisected tablet breakable into three portions; FIG. 2B shows a quadrisected tablet breakable into four portions; and FIG. 2C shows a pentasected tablet breakable into five portions.

FIGS. 3A-3F depict tablet scoring patterns, as in FIGS. 1A-1C and 2A-2C, respectively, as formed in bi-layer warfarin tablets in accordance with embodiments of the subject invention.

FIG. 4A is a cross-section of a taller than wide tablet looking towards the side of the tablet that has a score;

FIG. 4B is a cross-section of the tablet of FIG. 4a looking at the side of the tablet where the score ends.

FIGS. 5A-B and 5C-C are views of FIG. 4A and FIG. 4B respectively when the tablets have been broken through the score.

FIG. 6 is a cross-section of a taller than wide tablet having five segments.

FIGS. 7A-7B are views of FIG. 6 when the tablet has been broken through one segment.

FIGS. 8A-C are views of FIG. 6 when the tablet has effectively been broken through two segments in two steps, first by breaking the tablet and then by breaking the tablette of FIG. 7B.

FIG. 9 is an external perspective view of a scored tablet that has three segments.

FIG. 10 is an external view of a tablet with three segments on the middle segment of which are two horizontal (transverse) dotted lines close together.

FIG. 11 is a cross-section of a wider-than-tall tablet having two segments, one segment being deeply scored.

FIGS. 12A and 12B are views of FIG. 10 when the tablet has been broken at an angle from vertical.

FIG. 13 shows a cross-section of a three segment tablet, two segments being unitary segments.

FIGS. 14A and 14B depict two tablettes formed by splitting the tablet of FIG. 13 through the score.

FIG. 15A is an external perspective view of a three segment tablet with two unitary segments;

FIG. 15B is an en face external view of the same tablet shown in FIG. 15A.

FIG. 16 shows a tablet with four segments, two of which are unitary segments.

FIGS. 17A and 17B are cross-sections of each tablette formed by breaking the tablet of FIG. 16 through the middle segment.

FIGS. 18A and 18B demonstrate the tablettes formed by breaking the tablette of FIG. 17B as guided by the score.

### DETAILED DESCRIPTION OF THE INVENTION

The invention involves tablets, methods of dosing by breaking tablets, and related embodiments. Tablets of the invention can comprise at least two compositionally different layered segments.

In general, disclosure of a drug or drug product would also comprise the pharmaceutically acceptable salts, complexes, polymorphs, hydrates, isomers, or derivatives thereof. This inclusiveness need not be recited repetitively herein.

The subject invention includes pharmaceutical tablets comprising functional scores wherein such products have heretofore never included a functional score. This includes without limitation a functional bisecting score in a product previously only containing a decorative bisecting score, a functional trisecting score in a product previously only containing a decorative trisecting score, a functional quadrisecting score in a product previously only containing a decorative quadrisecting score, and the like.

These functional scores are scores that are dimensioned such that by breaking through the score, a partial tablet, or tablette, is provided that contains an accurate or predictable partial dose. The dimensions of a decorative score are typically less than about 1 mm in depth and are often less than a few millimeters in depth (depth refers to the vertical height of the score as produced in the die). The depth of the score is determined by the height of the embossing forming the score during compression; therefore an embossing that is raised 0.5 mm from the face of the die or punch contiguous therewith produces a 0.5 mm score in the tablet face. Functional scores, in accordance with the subject invention, are scores that are deeper than a conventional or decorative score when applied to the drug product. Often, a functional score is greater than 0.8 mm and often greater than 1 mm in depth. Depth is measured relative to the adjoining punch surface to the embossing. The punch surface may be flat relative to the orientation of the tablet press or may itself have a slant.

A functional score may be less than 0.8 mm. This depth (height) relates in large part to the tablet dimensions. A low weight tablet may have a highly functional score of only 0.5 mm in depth, using an embossing of 0.5 mm. If all prior versions of the tablet product of the invention used no more than a 0.15 mm deep score, for example, the invention would represent an advance in the art.

Furthermore, any tablet containing a score, preferably formed from an embossing on a tablet punch but not limited to such a method, which is about 1.0 mm or greater in depth (height) is novel and is part of the invention. Other non-limiting means of scoring tablets include use of a knife or rasp, and a novel printer-scoring machine that utilizes a high-speed cutting method.

A functional score of the subject invention is preferably provided in at least one layer of a tablet, and advantageously provides accurate and predictable partial doses in the resulting tablettes. A predictable and accurate partial dose in the resulting tablette is suggested to meet certain criteria, which may include one or more of the following:
i) a tablette containing a partial dose of a whole tablet has the same quantity of drug as a whole tablet containing the partial dose;
ii) each tablette containing a partial dose created by breaking the whole tablet as guided by a separation mark theron contains the same amount of drug as in another tablette containing the same partial dose; and/or
iii) after breaking a plurality of whole tablets into tablettes and grouping according to smallest, smaller, larger or largest sizes of resultant tablettes derived from breaking each tablet into smaller doses, each group of tablettes will have the same amount of drug as another group of tablettes.

The European Pharmacopoeia has instituted various factory release tests for uniformity of mass upon the breaking of scored tablets. No definitive international criteria, or FDA criteria, for a “functional” score is accepted. Furthermore, an important part of certain embodiments of the invention relate to the use of a tablet that breaks accurately with regard to dose (and perhaps to mass) is that said breaking be readily accomplished by a patient, including a patient of ordinary or less than ordinary skill in tablet breaking. In medical practice, trained tablet breakers such as pharmacists are usually not available when treating most patients, who are home-based and break their own tablets.

The amount of drug may be determined according to a standard pharmacopoeial uniformity of content assay. See United States Pharmacopocia, 2006 and European Pharmacopoeia, 2005 Edition, which are incorporated herein by reference.

Tablets of the invention are preferably produced on a layer press, such as a tri-layer or five-layer (e.g., TRP 900)high speed manufactured by Korsch AG of Germany. Remington's Pharmaceutical Sciences 20th Ed., Mack Publishing Co., Easton, Pa. (2000), Chapter 45, which is incorporated by reference, describes the various techniques utilized in making compressed tablets. The tablets of the invention are primarily intended for oral administration but they may also be used for other applications, such as through a gastrostomy feeding tube. Except for an excipient or drug having intrinsic adhesive properties used in a composition that is used to form a layer or segment, tablets of the invention are not formed using a cement, glue, adhesive, or the like. These tablets are typically not coated with a semipermeable membrane.

Certain embodiments of the subject tablets are formed from compressing, e.g., vertically with one composition placed on top of another, at least two different pharmaceutical formulation compositions, e.g., granulations, configured as separate layers or tablet segments; certain preferred tablets comprise three vertically disposed segments. Embodiments of the subject invention include, but are not limited to, a vertically compressed tablet having a height greater than its width (a “taller than wide” tablet), and a unitary segmented tablet. These embodiments can comprise a separation mark or score.

A layer is produced by introducing an amount of an individual granulation into a tablet die to fill at least a part of the die. A layer is considered to be present whether it is the form of an un-tamped, tamped or fully compressed granulation.

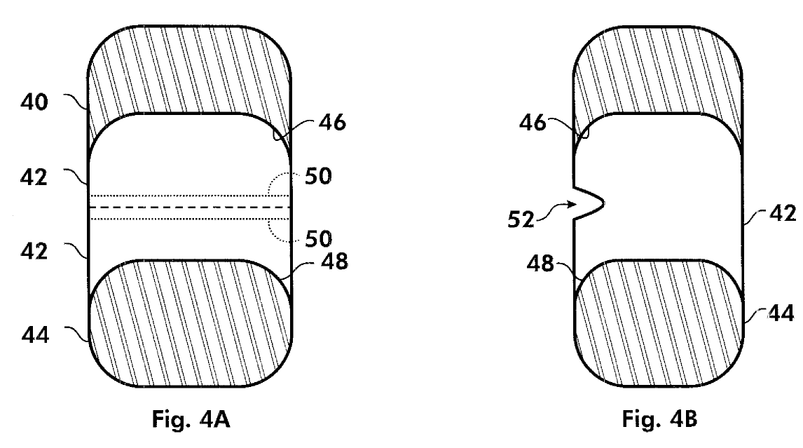

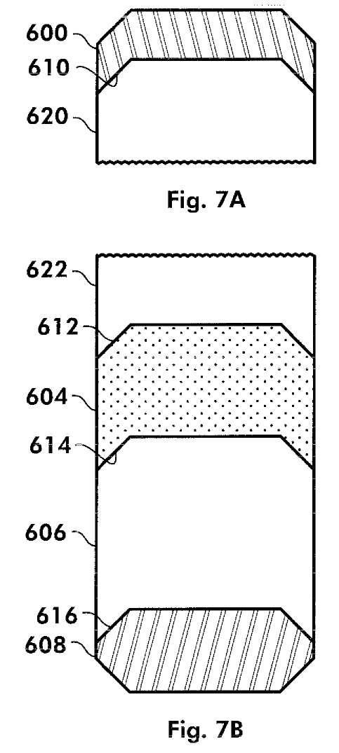

Examples of specific embodiments of the invention can be understood with reference to the drawings. The drawings depict perspective or vertical cross-sectional views of tablets and tablettes of the invention. Tablets are depicted in FIGS. 4-18 as if they were in the die, so that the top of the tablet as it is oriented on the page corresponds with the top of the tablet in the die. In other words, the top segment of the tablet as viewed contains the last granulation to enter the die. Tablettes are depicted as they would have been in the die before they were separated from the intact tablet. Shaded areas represent segments derived from active granulations, i.e., those which contain a drug; clear (plain) areas represent segments derived from inactive granulations, i.e., those formulated with no active drug.

Separation marks in the tablets depicted in the Figures as scores that are present on or in the surface of the tablet and that do not extend deeply enough into the tablet to appear in the cross-sectional front views are depicted in the drawings as dotted lines to reflect the location of said scores on or in the surface of the tablet (not shown). It is to be understood that the depth of a separation mark or other score may be deeper than one-half the widest cross-section of the tablet in a particular embodiment, and thus the transverse dotted lines reflecting scores that are separation marks shown in the Figures imply no intention to limit the depth of any scores of the tablets of the invention. Similarly, the tablets shown that contain scores do not limit the width or extent of said scores. The horizontal dotted lines on the front view that represent the surface scores are schematic, and do not necessarily represent the full vertical extent of the score. (Perforations or discontinuous scores through the width or depth of the tablets are not depicted herein, but remain within the scope of the invention, as are other marks on or physical changes to the tablet that create a separation mark). Any scores or printed indicia that serve as separation marks are for convenience herein assumed to be on the front surface of the tablet, which is arbitrarily chosen from a vertically-oriented surface of the tablets. The “side view” of a tablet is a cross-sectional view of the tablet rotated 90 degrees from the front view, and is shown, for example, in FIG. 4B. No dimension of the separation marks is limited by their depiction as dotted lines in any figure.

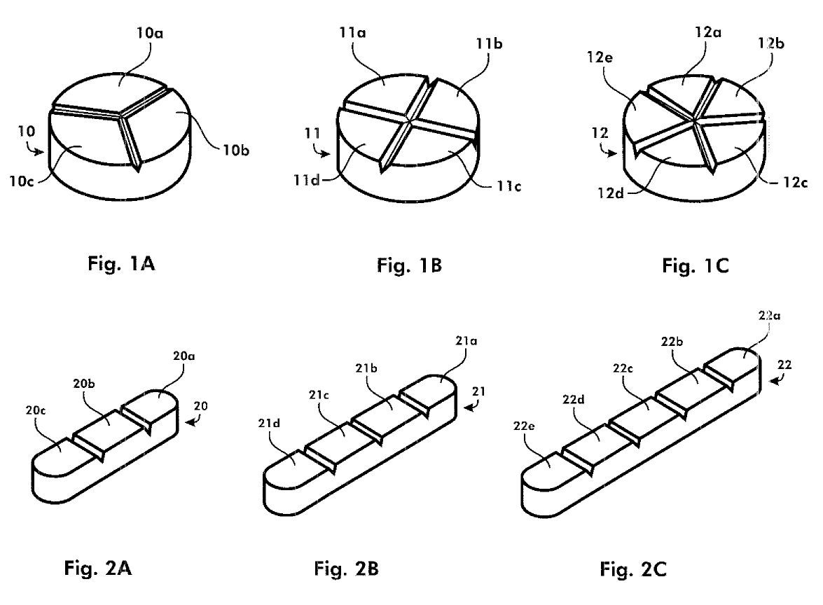

Turning to the Figures, FIGS. 1A-1C depict embodiments of cross-scored substantially round or ovoid warfarin tablets according to the subject invention. FIG. 1A shows a trisected tablet 10 containing a warfarin pharmaceutical composition, wherein the tablet is breakable along the score lines into one or more of the three portions 10a, 10b and 10c. FIG. 1B shows a quadrisected warfarin tablet 11 breakable along the score lines into one or more of the four portions 11a, 11b, 11c, and 11d. FIG. 1C shows a pentasected warfarin tablet 12 breakable along the score lines into one or more of the five portions 12a, 12b, 12c, 12d, and 12e.

FIGS. 2A-2C depict embodiments of substantially elongated, (capsule shaped) tablets according to the subject invention, comprising a plurality of parallel scores provided on the same surface of the tablet. In one embodiment of the invention, the tablet comprises warfarin or its pharmaceutically acceptable salt, e.g., warfarin sodium, as an active ingredient in the pharmaceutical tablet. FIG. 2A shows, for example, a trisected warfarin tablet 20 breakable along the score lines into one or more of the three portions 20a, 20b, or 20c. FIG. 2B shows a quadrisected warfarin tablet 21 breakable along the score lines into one or more of the four portions 21a, 21b, 21c, or 21d. FIG. 2C shows a pentasected warfarin tablet 22 breakable along the score lines into one or more of the five portions 22a, 22b, 22c, 22d, or 22e.

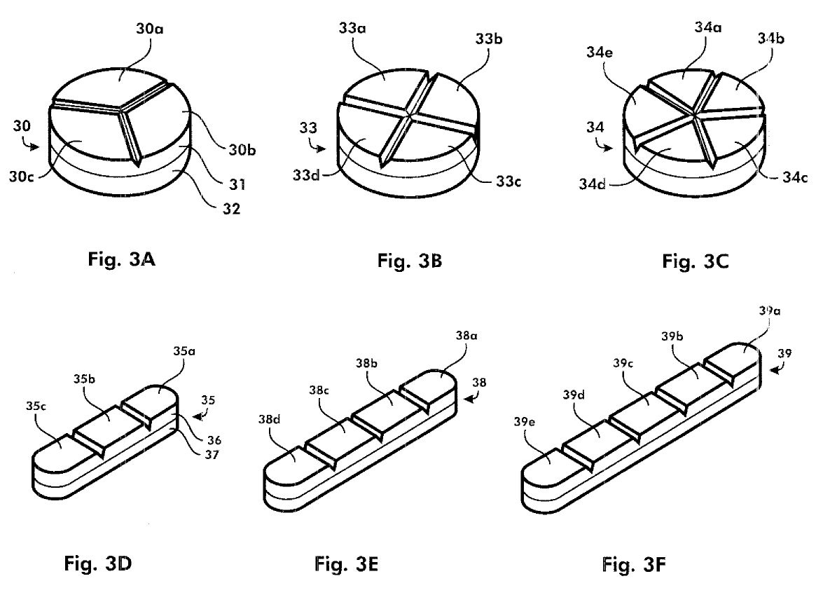

FIGS. 3A-3C depict tablet scoring patterns formed in substantially round or ovoid bi-layer warfarin tablets in accordance with the subject invention. FIGS. 3D-3F depict tablet scoring patterns formed in substantially elongate or capsule shaped bi-layer warfarin tablets in accordance with the subject invention. FIG. 3A shows a trisected bi-layer tablet 30 containing a warfarin pharmaceutical composition in a first layer 31 and an inactive composition in a second layer 32. The bi-layer tablet 30 is breakable along the score lines into one or more of the three bi-layer portions 30a, 30b or 30c. FIG. 3B shows a quadrisected warfarin bilayer tablet 33 breakable along the score lines into one or more of the four portions 33a, 33b, 33c, and 33d. FIG. 3C shows a pentasected warfarin bilayer tablet 34 breakable into one or more of the five portions 34a, 34b, 34c, 34d, or 34e. FIG. 3D shows a trisected bilayer tablet 35 containing a warfarin pharmaceutical composition in a first layer 36 and an inactive composition in a second layer 37, the bilayer tablet being breakable along the score lines into one or more of the three portions 35a, 35b, or 35c. FIG. 3D shows a quadrisected warfarin bilayer tablet 38 breakable along the score lines into one or more of the four portions 38a, 38b, 38c, or 38d. FIG. 3F shows a pentasected warfarin bilayer tablet 39 breakable along the score lines into one or more of portions 39a, 39b, 39c, 39d, or 39e.

FIGS. 4A and 4B depict a tablet with compositionally substantially identical upper segment 40 and lower segment 44. In a preferred embodiment, for example, a controlled-release formulation of a metoprolol salt is present in each segment. Inner segment 42 contains trace amounts of the drug that is present in a therapeutically effective quantity in each of segments 40 and 44; in a preferred embodiment said drug comprises coated particles of metoprolol. Interfaces 46 and 48 represent regions in which the upper part of segment 42 and the lower part of segment 42 respectively adjoin upper segment 40 and lower segment 44. The curved interfaces result from the profile of the upper tablet punch which is curved. Score 52 is depicted in FIG. 4B. Dotted line 50 in FIG. 4A is a reflection of score 52 on the surface of the tablet (not shown), that does not penetrate half-way through the shorter transverse axis of the tablet.

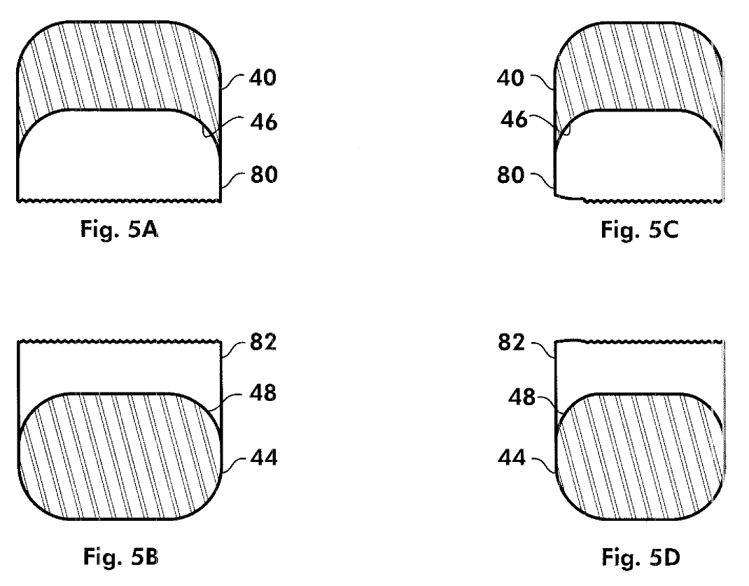

FIGS. 5A-D depict tablettes formed from breaking the tablet of FIGS. 4a and 4b through score 52. Inner segment 42 of FIG. 4A no longer exists as an intact segment. The upper tablette of FIGS. 5A and 5C contains segment 80 that adjoins intact upper segment 40 and the lower tablette contains segment 82 and intact segment 44.

Breaking the tablet of FIGS. 4A and 4B through the score placed in segment 42 is clearly easier than breaking the tablet through its vertical dimension, which is currently the practice with scored layered (segmented) tablets, though it should be noted that the current and limited practice of scored layered tablets involves, probably exclusively, tablets that are taller than they are wide. The fact that during preferred means of breaking said tablet, no break is made in the parts of the tablet where the active drug has been placed provides for exceptionally accurate breaking relative to the active drug or drugs contained in the tablet.

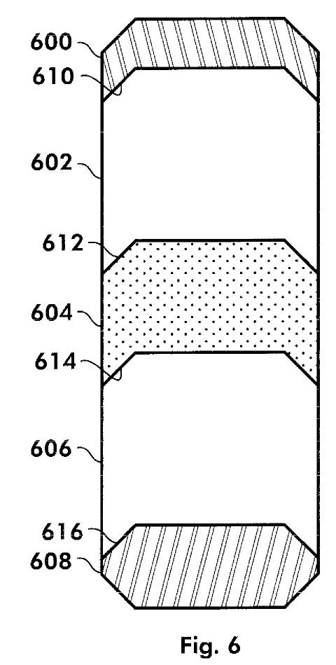

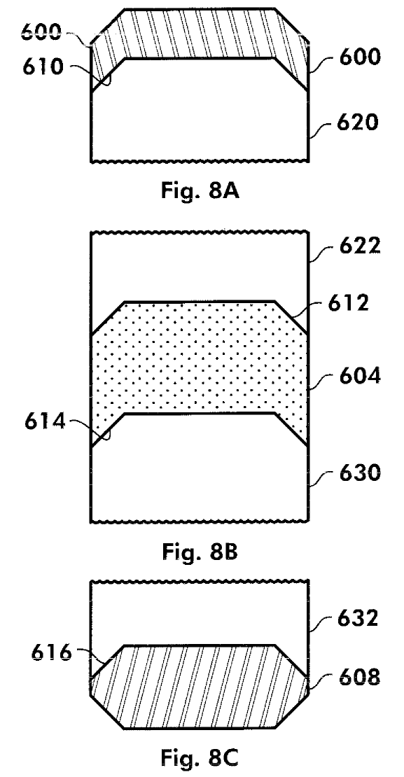

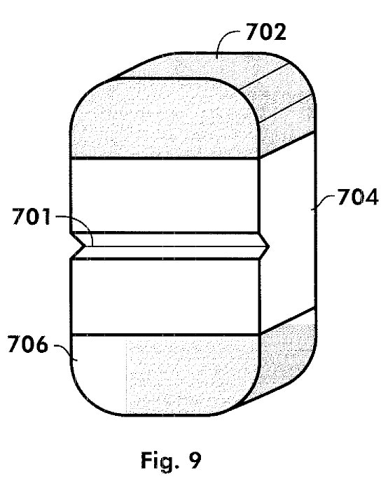

FIG. 6 illustrates a tablet more elongated than those previously demonstrated. Said tablet is adapted, even more than the others, for ease of breaking through one segment. Upper segment 600 is provided with a therapeutic quantity of a drug; stippled inner segment 604 is provided with a therapeutic quantity of a different drug; and, lower segment 608 is provided with a therapeutic quantity of a drug different from that found in a therapeutic quantity in segments 600 and 604. Clear (plain) inner segments 602 and 606 contain pharmacologically ineffective amounts of each of the three drugs found in the tablet, though in less preferred embodiments, inadvertent mixing of the three different granulations is sufficient to produce a pharmacologically effective though not therapeutically effective dose of a drug or drugs. In yet a different embodiment, segment 604 is provided with a therapeutic quantity of a vitamin such as folic acid; and in addition, in a less intact segment. Assuming minimal to no intermixing between the materials forming each segment of the tablet of FIG. 6 and assuming that segment 632 of FIG. 8C is substantially free of any active drug, the tablette of FIG. 8C represents a novel altered release dosage form, in part in that it consists of an altered release product adjoining in a segment an immediate release substantially inactive segment.

FIG. 9 is a perspective view of a tablet of the invention which shows score 701 as a separating mark on a front surface and top active (drug-containing) segment 702; middle pharmacologically inactive segment 704 (no pharmacologically effective amount of a drug) and bottom active segment 706. When the tablet is broken through the score 701, the top segment and the bottom segment will remain intact. Segments 702 and 706 each contain a compositionally identical controlled release beaded formulation of one or more active pharmaceutical ingredient, e.g., verapamil plus immediate-release hydrochlorothiazide (“HCTZ”). Pharmaceutically ineffective quantities of verapamil and HCTZ may be found in segment 704.

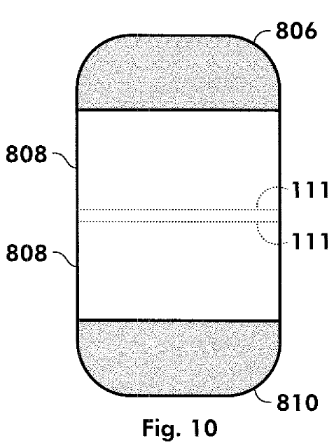

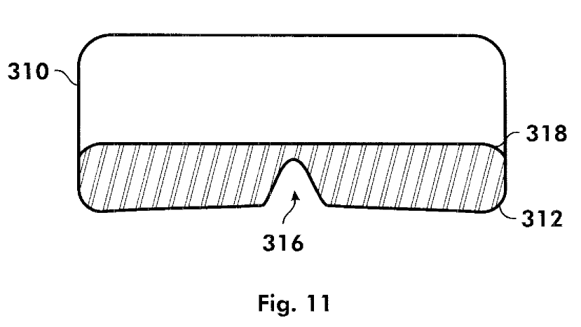

FIG. 10 shows a front view of a tablet according to the invention that has two printed dotted lines that serve as a separation mark according to the invention. Middle segment 808 comprises inactive excipients only or a therapeutically effective quantity of, e.g., folic acid and vitamin B12. Lower segment 810 comprises controlled release beads of, e.g., a metoprolol salt in a therapeutic quantity and upper segment 806 comprises a therapeutic quantity of amlodipine. Sub-therapeutic quantities of the following drugs are found in the following segments: folic acid and vitamin B12 in segments 806 and 810, metoprolol in segments 806 and 808, and amlodipine in 808 and 810. FIG. 11 depicts an immediate release tablet with a score 316 that extends approximately 90% through the bottom segment 312. Upper segment 310 allows structural stability of the tablet despite the deep score 316. In this tablet, no pharmacologically effective dose of the drug present in segment 312 is present in segment 310. In another preferred embodiment, segment 310 may contain a different drug than is present in segment 312, preferably in a pharmacologically effective quantity. In another preferred embodiment, segment 310 contains a pharmacologically effective quantity of the drug or drugs present in segment 312, but in a lesser concentration relative to the excipients in each segment. Interface 318 is present. In this tablet, an active drug in a composition with altered release characteristics is present in a therapeutic quantity in segment 312, and segment 310 lacks a therapeutic quantity of the same or any other drug. The novelty of the tablet design would remain, however, were segment 310 to be provided with drugs such as an effective amount of a different drug. Novelty would also be present, for example, if the altered release composition were present in a therapeutically effective quantity in unscored segment 310 and a therapeutically effective quantity of a different immediate release composition of a drug were present in scored segment 316.

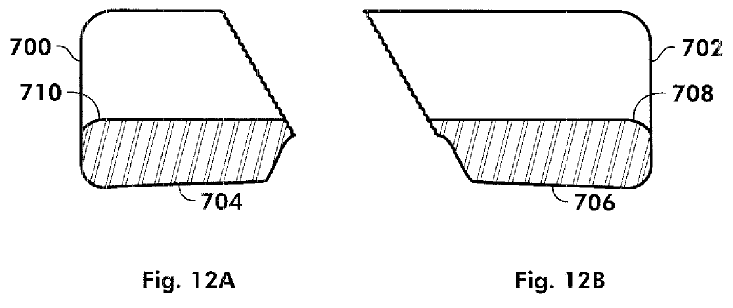

Breaking the tablet of FIG. 11 may give two tablettes as shown in FIGS. 12A and 12B, though no limitation as to the direction of tablet breaking is intended. Largely inactive segment 310 of FIG. 11 has been divided into two segments, 700 in the smaller tablette as shown in FIG. 12A and 702 in the larger tablette of FIG. 12B. Even though breaking as demonstrated is far from vertical, it is clear that the amount of drug in new segments 314 and 315 created from segment 312 of FIG. 11 is similar. Two new segments, 706 in FIG. 12B and 704 in FIG. 12A, are created by said creation of the two tablettes. New interfaces 708 and 710 lie at the regions at which segments 702 and 706, and 700 and 704, respectively, adjoin.

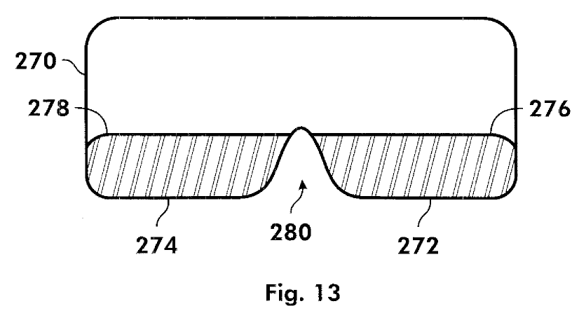

FIG. 13 depicts a tablet containing unitary segments 272 and 274 in vertical cross-section, front view. Both of said unitary segments adjoin the same face (surface) of segment 270, which is formed from a single granulation and due to mixing of granulations, contains a minimal amount of the drug that is present in segments 272 and 274. Interfaces 276 and 278 represent the regions at which segment 270 adjoins segments 272 and 274, respectively. Score 280 indents segment 270 and also represents the space between segments 272 and 274. Unitary segments 272 and 274 contain an altered release composition of a pharmaceutical. Segment 270 is formed from inactive excipients that do not affect the release rate of said pharmaceutical from the tablet.

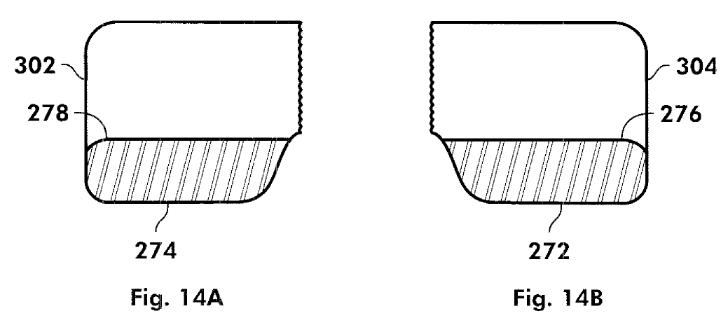

FIGS. 14A and 14B depict the two tablettes created by breaking the tablet of FIG. 13 through segment 270. In FIG. 14A, segment 302 represents that part of segment 270 that adjoins intact segment 274. Interface 278 represents the region at which segments 302 and 274 meet. In FIG. 14B, interface 276 represents the region at which segments 304 and 272 meet. Score 280 and segment 270 of FIG. 13 are not considered to exist once the tablettes are formed. Each tablette of FIGS. 14A and 14B contains substantially equivalent mass assuming the score 280 of FIG. 13 is a bisecting score relative to the layer that became divided in the creating of segments 272 and 274.

Tablets of the nature of that of FIG. 13 may contain in the unitary segments a mixture of drugs or one drug. In addition, the granulation that forms segment 270 of FIG. 13 may be provided with a drug that is the same as, or different than, that of the divided layer. In such a case, it would be preferable that said drug provided in the upper layer (segment) would have a therapeutic effect and side effect profile that was not very sensitive to accuracy of subdivision of a dose.

In addition, no limitation exists as to the presence of one or more additional segments created superior to (i.e., above) segment 270, or the composition of such. Also, though less likely, there could be another set of different unitary segments inferior to (i.e., below) segments 272 and 274.

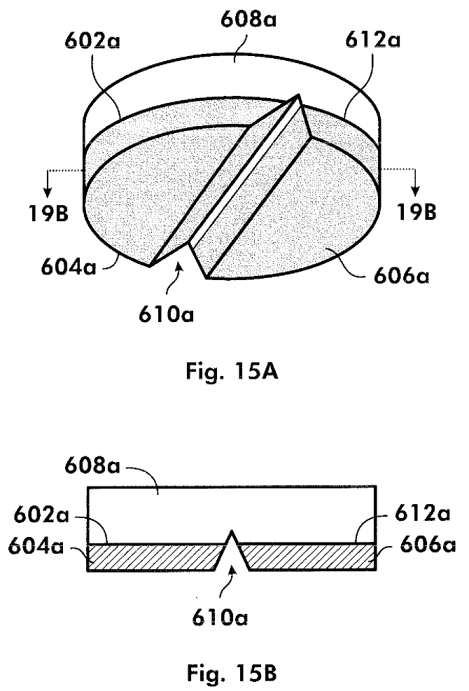

FIG. 15A depicts an external view of a tablet containing unitary segments 604a and 606a that are at the bottom of the tablet. In this tablet, score 610a penetrates into clear, upper, non-unitary segment 608a. Interface 602a represents the region at which segment 608 meets segment 604a. Interface 612a represents the region at which segment 606a meets segment 608a.

FIG. 15B depicts the same tablet depicted in FIG. 15A. This vertical cross-section is taken perpendicularly through score 610a, which occupies the diameter of the circular transverse cross-section of the tablet. The unitary segments of the tablets of FIGS. 15A and 15B comprises therapeutic quantities of diltiazem in beads producing controlled release of diltiazem to last twelve hours at least in therapeutic effect. Segment 608a has no therapeutically effective quantity of any drug.

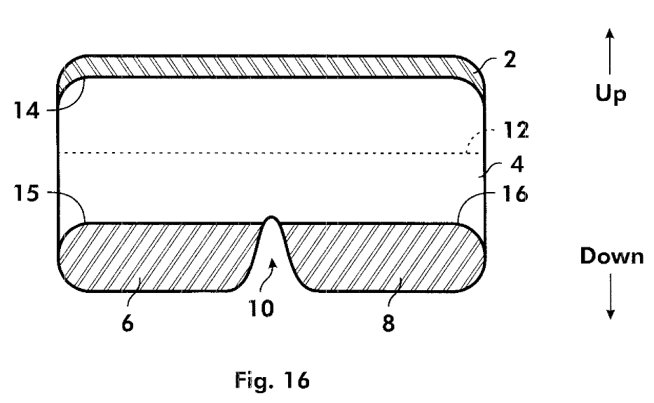

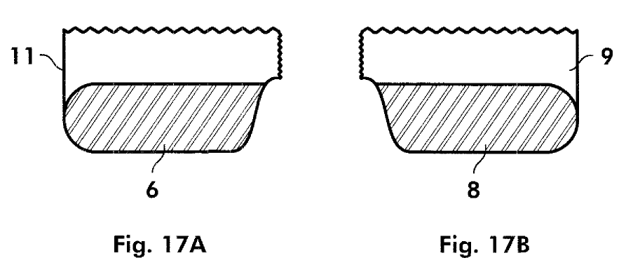

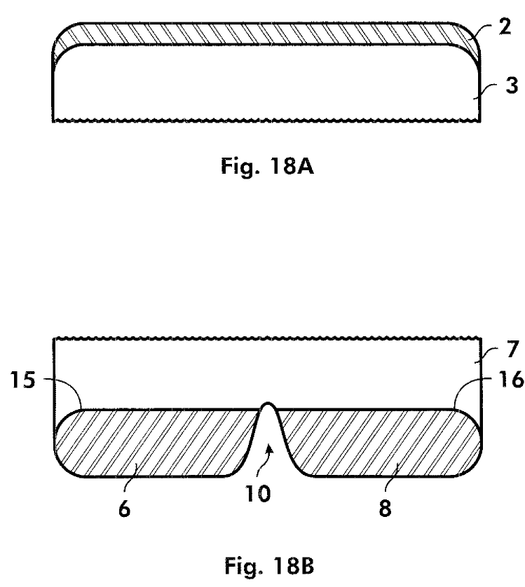

FIG. 16 depicts a tablet containing four segments. Unitary segments 6 and 8, as with all unitary segments, are not contiguous with each other. Score 10 penetrates into segment 4. Segment 4 is a compound segment formed from substantially compositionally identical inactive granulations added sequentially with immediate release characteristics. Top segment 2 contains a therapeutic quantity of a drug that differs from the drug that is present in a therapeutic quantity in segments 6 and 8. Dotted line 12 reflects a surface score that runs transversely across segment 4. A preferred horizontal dimension for the tablet of FIG. 16 is 12-18 mm, but said dimension is not limited. Interface 14 depicts where segments 2 and 4 are contiguous. Interfaces 15 and 16 depict where segments 6 and 8, respectively, adjoin segment 4. Segment 4 contains therapeutically insignificant quantities of the drugs found in segments 6 and 2. The tablet of FIG. 16 may be broken usefully in two ways. One way is vertically through score 10 in the direction of segment 2, to provide tablettes as shown in FIGS. 17A and 17B; such breaking would not utilize the score reflected by dotted line 12, but would give a dose of half of the drug found in segments 6 and 8, though likely would not give a precise halving of the drug found in segment 2, due to difficulties with breaking scored tablets as was documented in the Background of the Invention, above. The result of another way of breaking said tablet (horizontally) is depicted schematically in FIGS. 18A and 18B.

As an example of a method of manufacture of a preferred tablet of the invention, first, a granulation containing a pharmacologically effective dose of a drug enters the die and is tamped to form a first segment. Second, a granulation lacking a drug (an “inactive granulation”) enters the die and is tamped. The inactive granulation creates a part of the tablet that can be identified and broken through so that a part of the drug containing a significant concentration of drug is not broken through. Last, a second granulation containing a pharmacologically effective quantity of a drug enters the die, is optionally tamped, and then final compression to form a third segment and a final compressed tablet occurs. While one or all segments may individually have a width greater than height, the tablet as a whole preferably has a height that exceeds its width.

Especially suitable dimensions for tablets for human use according to the invention are, without limitations, the following: height: 6 to 24 mm; preferable 10 to 18 mm and more preferably from 10 to 14 mm; width (at the widest dimension of the horizontal axis): 2 to 16 mm; preferable 3 to 10 mm and more preferably 4 to 8 mm. Without limitations, the dimensions of the tablet may be optimal if the ratio of the height to the width is between about 1.5:1 to about 3:1.

Subsequent to tablet formation, optionally a score may be placed in the side of said tablet, preferably transversely (horizontally). Alternatively, after tablet formation, a printed line or other forms of indicia such as dotted lines, symbols or perforations may be placed on or in the surface of the tablet, all of which serve the purpose of allowing identification of said tablet's desired breaking region from the standpoint of effecting accurate separation of the parts of a tablet containing isolated doses of drug. Other means of aiding identification of a region of potentially desired tablet breaking may be utilized such as the use of contrasting colors in different segments.

Additionally, the compressed tablet can be further processed to provide an inert covering or container, e.g., a capsule or a sachet. In use, the covering can be cut away or otherwise removed, such as by twisting apart or carefully cutting a conventional gelatin capsule, removing the tablet therein and dividing the tablet as described herein for a non-encapsulated embodiment. The covering or container can advantageously be useful to minimize or prevent confusion on the part of the patient user viewing a segmented or layered tablet of the subject invention.

In certain of the preferred tablets of the invention, a layer (and the granulation from which it is derived) will not need to be placed on top of or below (e.g., adjoining, or contiguous with) a substantially identical layer (or granulation). In such a case, one layer will give rise to the sub-type of segment that is a simple segment. The use of the term “segment” allows a segment to be simple or compound. Because the tablets of the invention have been adapted to be broken if and when desired, a term for the major fragments resulting from said breaking has been coined. The inventors use the term “tablette” in this regard.

An example of tablette formation is as follows: a standard single-scored, mono-layer, homogeneous pharmaceutical tablet is broken through the score to produce two “half” tablets. Each of said major fragments is called a tablette. Some chipping and crumbling, which are preferably minor in amount, may occur; these chips, crumbs etc. are not considered to be tablettes. It is often advantageous to place a score transversely into a segment, such as a substantially inactive segment that is not a top end segment (and therefore is an “inner” segment), as may be done with an instrument such as a file or a saw-like process. Successfully breaking said tablet through said score is designed to create two tablettes, representing the two major fragments of the tablet and not including smaller fragments such as crumbs or chips. The score or non-score separation mark such as printed indicia need not be placed in the middle of the height of the tablet, and therefore the tablettes may be intended to be of substantially different height and may be a 2:1 or 3:1 height ratio, for example.

Of the many tablets that can be produced according to the invention, examples of a tablet manufactured in a multilayer tablet press are as follows:

One embodiment comprises a pharmaceutical tablet having compositionally substantially identical first and second “unitary segments” that each adjoin the same face (surface) of a compositionally distinct first non-unitary segment. Said pharmaceutical tablets preferably comprise two or more compositionally identical unitary segments including a first unitary segment and a second unitary segment, said first unitary segment and said second unitary segment containing a drug or drugs, said first and second unitary segment having been formed from the same layer or layers that was or were divided; said first segment optionally having a score on its surface positioned between said first and said second unitary segments; said tablet optionally having additional unitary segments; and said tablet having at least one segment that is not a unitary segment. This embodiment of the invention is illustrated in FIGS. 15A and 15B, and is described in detail in WO 2006/038900, which is hereby incorporated by reference in its entirety.

The term “unitary segment” means a physically separated, non-contiguous part of a divided layer or layers of a tablet and which may be made using a bottom embossed die that causes granulate to be divided as it enters the tablet die or after compression by an upper punch in the die; or, by a post-tabletting scoring that removes a part of a segment to a depth that exposes an underlying segment.

A layer is produced by introducing an amount of an individual granulation into a tablet die to fill at least a part of the die. A layer is considered to exist whether it is in the form of an un-tamped, tamped or fully compressed granulation. Because some migration of granulation may take place in the tabletting machine, some amount (preferably of no therapeutic importance) of a granulation that forms one layer may be transferred to another layer.

This unitary segment embodiment may have one or more additional unitary segments in addition to said first and second unitary segments that are optionally present and that are derived from the same layer or layers as said first and second unitary segments.

A preferred method of making this unitary segment embodiment of a tablet of the invention utilizes a protuberance known as an embossing that rises from the lower punch of a tablet die in a tabletting machine. In a preferred method of manufacturing, a granulation preferably containing a therapeutic amount of an active pharmaceutical ingredient enters the die, preferably forms a layer above the highest point of said embossing, and is tamped by the upper punch. Next, a second granulation that is different from said first granulation enters said die on top of said first granulation, preferably is tamped by the upper punch, and then the tablet is compressed by the upper punch so that said compression pushes said first granulation below the highest points of said embossing. In the invention, said embossing occupies a position on the lower punch that may bisect or quadrisect said lower punch, so that said compression causes said first layer to be divided into two or more non-contiguous segments. Said first layer formed from said first granulation is herein referred to as a divided layer; said segments formed from a divided layer are herein referred to as unitary segments. The invention therefore allows precise division of said tablet, when desired, by allowing breaking to occur only through the second layer that was formed by said second granulation so that maximal accuracy of dosing with a tablet fragment arising from intentional tablet breaking may occur. In the above example, said second granulation preferably lacks an active drug (i.e., it is an inactive granulation).

An alternative embodiment concerns a variation of the unitary segment tablet, but where the embossing does not completely separate the active layer into separate segments; rather, the embossing forms a deep score into the active segment, which is often formed from a single layer and is therefore often a simple segment. This embodiment of an accurately breakable dosage form of the subject invention is illustrated in FIG., and is described in detail in WO 2006/038916, which is incorporated by reference in its entirety.

Another embodiment of an accurately breaking dosage form of the subject invention is manufactured as follows: a first granulation comprising drug A enters into a die at a first filling station; a second granulation comprising inactive excipients enters on top of said first granulation at a second filling station; a granulation substantially identical in composition and quantity (weight) to said first granulation enters at a third filling station. After final compression, said tablet is ejected from the die. Each granulation, upon full entry into the die and thereafter, forms a layer, or segment, of the final tablet product. Appropriate tamping of each layer following each fill of a composition may be formed according to known or otherwise appropriate processes. Other procedures standard or appropriate for layered tablets are performed. There is no absolute requirement that the compositions be identical even if the drugs are identical. Different excipients may be used, and different colorings in the different drug-containing layers can be used. In certain preferred embodiments, a drug can be present in one of the active compositions in an immediate release composition, and the same drug can be present in the other active composition in a controlled release composition, such as a matrix or micro-particulate composition. In any of the active (drug-containing) tablets of the invention, the same drug may be present in the same quantity in different layers (segments) or in different quantities.

Preferably, in any of the manufacturing processes employed to form a tablet of the subject invention, there is no mixing of drug or excipients from one segment to another. However, in reality, minimal, inadvertent mixing between different granulations in the formation of layers can occur. Therefore, some mixing is to be expected and does not alter the improvement in the art of creating accurate dosing from breakable tablets from the invention. Different granulations may be of the same or different colors. Wet granulations are often preferred to limit transfer of material from one granulation to another. Direct compression of powder is also a preferred manufacturing technique.

By convention herein, the term “segments” may be place of “layers” in general in discussing the finished tablets of the invention, for reasons that are explained below. In addition, for convenience of reference and consistency throughout this specification, the descriptions herein may refer to the segments as comprising or utilizing a particular “granulation”. Such term is not limited to the formation of granules, per se, as in a wet granulation process. Other formulation compositions, for example, homogeneous mixtures or blends used in direct compression matrix formulations, coated or uncoated beads or pellets used in compressed tablets, or like compositions as are well known in the art and suitable for use in conventional layered, compressed tablet technologies, can be readily substituted for such “granulations” and are considered within the scope of the invention. It is expressly intended that the subject invention include each of these alternatively available and well known compressible formulation technologies.

A segment represents the entirety of a substantially homogeneous contiguous part of a tablet. A segment may be formed from more that one layer, however. If two substantially identical granulations entered the tablet die successively, with the second entering directly after and onto the first, such as at two successive filling stations during automated high-speed tablet manufacture, then the two granulations would each form a separate layer after entering, but when compressed, they would comprise one segment. A segment therefore is a basic unit of how the tablets of the invention prove useful. If, however, two different active drugs, or different salts of the same active drug, were compressed one on top of the other, they would form two segments. Granulations comprising the same active drug but with dissimilar excipients would also form two segments if one granulation were compressed onto another.

A segment formed by a plurality of layers that are formed from substantially identical granulations is called a compound segment. Compound segments may prove useful in situations of relatively large quantities of an inactive granulation, or granulation containing a drug or drugs, so that two or more consecutive fills (“feeds”) of substantially identical granulation may occur.

A layer formed from a granulation that is neither disposed upon nor under (i.e., does not adjoin and is not contiguous with) a substantially identical granulation is a simple segment. A non-compound segment is a simple segment.

As used herein, such terms as “horizontal” (“transverse”) and “vertical” when used in relation to a tablet, are based on the spatial orientation of the tablet as, and after, it is produced in a die, but before removal or ejection from the die. Current methods of manufacture produce tablets with one granulation entering the die on top of another, so that tablets of the invention produced in such a manner comprise one or more top (outer) segments, one or more bottom (outer) segments, and optionally one or more middle (inner) segments. A segment that is not a top or bottom (i.e., outer) segment is considered to be an inner segment.

In any configuration of a tablet according to the subject invention, the lateral parts of any outer or inner segment have an externally exposed surface.

If separate granulations were to be sequentially placed in a horizontally (side-to-side) and not vertically as is currently the practice, then the tablets so produced would be within the scope of the present invention because the same resultant product would be produced by the horizontal compression process. When the tablet of FIG. 1, for example, is laid on a flat table, it will tend to lie lengthwise at right angles to the manner in which it is formed in the die, so that if the three segments were all different colors, then the segments would appear to be arranged not vertically (one on top of the other), but rather horizontally (side-to-side). For consistency of terminology, such segments nonetheless are considered herein to be disposed vertically on top of each other.

Tablets of the invention are preferably uncoated, but can be coated with conventional coatings for aesthetic or functional or other purpose. However, these coatings are not regarded as a “layer” or “segment” of the tablets of the subject invention. These coatings do not significantly alter the release kinetics of the drug or drugs of the tablets of the invention.

The terms “active agent,” “drug,” “active drug,” “active pharmaceutical agent,” “pharmacologically active agent,” “active pharmaceutical ingredient” and the like are interchangeable and include, without limitation, prescription and non-prescription pharmaceutical compounds, as well as pharmacologically effective doses of vitamins, cofactors, and the like.

The following list of possible combinations of a plurality of drugs is exemplary and not limiting.

The combinations referred to may include two or more members of the classes listed. Drugs listed below, and herein, may for convenience exclude mention of any salt of a drug; e.g., “atorvastatin” is listed even though its marketed form is atorvastatin calcium.

Without limitation, useful combinations may include a plurality of drugs from within the following six drug classes. In addition, tablets of the invention may be created containing only one drug from the following list. With regards to combination use, two methods of use may apply to the invention. One of these methods is to place an individual drug in a granulation and a different individual drug (or combination of drugs) in a different granulation, potentially with an inactive granulation interposed between them; another method is to place a plurality of drugs in one or more segments.

Examples of drugs that may be used in the invention are, without limitation, the following. Where drug classes are provided, the entirety of the class is intended, with examples given.

1. Anti-anginal agents, for example:
A. Calcium antagonists (see list below);
B. Beta-blocker (see list below);
C. Organic nitrate preparation (e.g., isosorbide mononitrate or dinitrate).

2. Anti-anginal agent plus an anti-platelet agent, such as aspirin, clopidogrel, or ticlopidine.

3. Two hypoglycemic agents (see list below).

4. Potassium chloride and any thiazide-type or loop diuretic (see lists below).

5. Lipid-lowering agent plus: hypoglycemic agent, anti-platelet agent, anti-anginal agent, and/or antihypertensive agent (see lists above and below)

Hypoglycemic agents include: thiazolidinediones: pioglitazone, rosiglitazone; sulfonylureas: glyburide, glipizide, glimepiride, chlorpropamide; Biguanides: metformin; Meglitinides: nateglinide, repaglinide; Glucosidase inhibitors: acarbose, miglitol.

6. Antihypertensive agents: Beta-blockers: acebutolol, atenolol, bisoprolol, celiprolol, metoprolol, mebivolol, carvedilol (a mixed alpha-beta blocker), nadolol, oxprenolol, penbutolol, pindolol, propranolol, timolol, betaxolol, carteolol;

Calcium antagonists (calcium-channel blockers): nifedipine, amlodipine, verapamil, diltiazem, nisoldipine, felodipine, isradipine, lacidipine, lercanidipine, nicardipine, manidipine, aranidipine, atosiban, azelnidipine, barnidipine, belfosdil, bisaramil, buflomedil, cilnidipine, clentiazem, clevidipine, darodipine, diperdipine, dopropidil, dotarazine, dronedarone, efonidipine, elogodipine, emopamil, etomoxir, fantofarone, fasudil, furnidipine, iganidipine, ipenoxazone, lemildipine, leualacin, lifarizine, lomerizine, lubeluzole, mibefradil, monatepil, nefiracetam, nexopamil, Nifelan, nilvadipine, nimodipine, oxodipine, palonidipine, pranidipine, ranolazine, semotiadil, siratiazem, tamolarizine, temiverine, terodiline, vexibinol, zatebradine, and ziconotide.

Thiazide-type diuretics (with or without potassium-retaining diuretics such as triamterene, amiloride, or spironolactone): hydrochlorothiazide, chlorothiazide, cyclopenthiazide, polythiazide, bendrofluazide, hydroflumethiazide, chlorthalidone, indapamide, methylclothiazide, metolazone;

Angiotensin converting enzyme inhibitors: captopril, enalapril, lisinopril, ramipril, trandolapril, quinapril, perindopril, moexipril, fosinopril;

Angiotensin receptor blockers: losartan, valsartan, candesartan, telmisartan, eprosartan, irbesartan;

High-ceiling (loop) diuretics (with or without potassium-retaining diuretics such as triamterene, amiloride, or spironolactone): furosemide, torsemide, ethacrynic acid, bumetamide;

Aldostcrone antagonist diuretics: spironolactone, eplerenone;

Alpha-blockers: doxazosin, terazosin, prazosin, indoramin, labetolol (a mixed alpha-beta blocker);

Central alpha-agonists: clonidine, methyldopa;

Imidazoline: moxonidine;

Direct vasodilators: hydralazinc, minoxidil;

Adrenergic neuronal blocker: guanethidine.

Lipid-lowering agents include:
Statins: lovastatin, simvastatin, pravastatin, rosuvastatin, atorvastatin, fluvastatin;
Fibrates: clofibrate, bezafibrate, fenofibrate, gemfibrozil, ciprofibrate;

Others: ezetimibe, niacin, acipimox, ciglitazone, englitazone, acarbose, miglitot, voglibose, repaglinide, gliquidone, nateglinide, gliclazide, glimepiride, aliskiren, remikiren, zolasartin, cilexitil, tasosartan, fentiapril, pivalopril, zoefenopril, alacepril, amalopril, spirapril, indolapril, pentopril, cilazapril, imidapril, and dronedarone

The drugs disclosed herein are for illustrative purposes and are not intended to limit the scope of the invention. Any available drug that can be tabletted may be used in accordance with the subject invention. Such drugs are listed in The Physicians' Desk Reference (PDR), Secions 2 and 3, 2006 Edition, which lists numerous classes and names of drugs, as well as the European Pharmacopoeia (EP). The relevant portions of the PDR and EP, e.g., the listing of drug substances that can be used in accordance with the subject invention, are incorporated herein in their entirety by reference. A copy of Section 2 (Brand and Generic Name Index) and Section 3 (Product Category Index) of the PDR, as well as an Alphabetical Index of drug substances are attached as an Appendix hereto. These drugs and drug categories are considered to be within the scope of the invention, and are exemplary only, as other drugs useful for treating humans or animals, but not included in one or more of the attached lists, may also be used in accordance with the subject invention.

A partial list of drug substances useful in accordance with the subject invention, including controlled substances, are listed below:

| CONTROLLED SUBSTANCE | SYNONYM |
|---|---|
| 1-(1-Phenylcyclohexyl)pyrrolidine | PCPy, PHP, rolicyclidine |
| 1-(2-Phenylethyl)-4-phenyl-4-acetoxypiperidine | PEPAP, synthetic heroin |
| 1-[1-(2-Thienyl)cyclohexyl]piperidine | TCP, tenocyclidine |
| 1-[1-(2-Thienyl)cyclohexyl]pyrrolidine | TCPy |
| 13Beta-ethyl-17beta-hydroxygon-4-en-3-one | |
| 17Alpha-methyl-3alpha,17beta-dihydroxy-5alpha- | |
| androstane | |
| 17Alpha-methyl-3beta,17beta-dihydroxy-5alpha- | |
| androstane | |
| 17Alpha-methyl-3beta,17beta-dihydroxyandrost-4- | |
| ene | |
| 17Alpha-methyl-4-hydroxynandrolone (17alpha- | |
| methyl-4-hydroxy-17beta-hydroxyestr-4-en-3-one) | |
| 17Alpha-methyl-delta1-dihydrotestosterone (17beta- | 17-Alpha-methyl-1-testosterone |
| hydroxy-17alpha-methyl-5alpha-androst-1-en-3-one) | |
| 19-Nor-4-androstenediol (3beta,17beta- | |
| dihydroxyestr-4-ene; 3alpha,17beta-dihydroxyestr-4- | |
| ene) | |
| 19-Nor-4-androstenedione (estr-4-en-3,17-dione) | |
| 19-Nor-5-androstenediol (3beta,17beta- | |
| dihydroxyestr-5-ene; 3-alpha,17beta-dihydroxyestr-5- | |
| ene) | |
| 19-Nor-5-androstenedione (estr-5-en-3,17-dione) | |
| 1-Androstenediol (3beta,17beta-dihydroxy-5alpha- | |
| androst-1-ene; 3alpha,17beta-dihydroxy-5alpha- | |
| androst-1-ene) | |
| 1-Androstenedione (5alpha-androst-1-en-3,17-dione) | |
| 1-Methyl-4-phenyl-4-propionoxypiperidine | MPPP, synthetic heroin |
| 1-Phenylcyclohexylamine | PCP precursor |
| 1-Piperidinocyclohexanecarbonitrile | PCC, PCP precursor |
| 2,5-Dimethoxy-4-(n)-propylthiophenethylamine | 2C-T-7 |
| 2,5-Dimethoxy-4-ethylamphetamine | DOET |
| 2,5-Dimethoxyamphetamine | DMA, 2,5-DMA |
| 3,4,5-Trimethoxyamphetamine | TMA |
| 3,4-Methylenedioxyamphetamine | MDA, Love Drug |
| 3,4-Methylenedioxymethamphetamine | MDMA, Ecstasy, XTC |
| 3,4-Methylenedioxy-N-ethylamphetamine | N-ethyl MDA, MDE, MDEA |
| 3Alpha,17beta-dihydroxy-5alpha-androstane | |
| 3Beta,17beta-dihydroxy-5alpha-androstane | |
| 3-Methylfentanyl | China White, fentanyl |
| 3-Methylthiofentanyl | Chine White, fentanyl |
| 4-Androstenediol (3beta,17beta-dihydroxy-androst-4- | 4-AD |
| ene) | |
| 4-Androstenedione (androst-4-en-3,17-dione) | |
| 4-Bromo-2,5-dimethoxyamphetamine | DOB, 4-bromo-DMA |
| 4-Bromo-2,5-dimethoxyphenethylamine | 2C-B, Nexus, has been sold as Ecstasy, i.e. MDMA |
| 4-Dihydrotestosterone (17beta-hydroxyandrostan-3- | Anabolex, Andractim, Pesomax, Stanolone |
| one) | |
| 4-Hydroxy-19-nortestosterone (4,17beta- | |
| dihydroxyestr-4-en-3-one) | |
| 4-Hydroxytestosterone (4,17beta-dihydroxyandrost- | |
| 4-en-3-one) | |
| 4-Methoxyamphetamine | PMA |
| 4-Methyl-2,5-dimethoxyamphetamine | DOM, STP |
| 4-Methylaminorex (cis isomer) | U4Euh, McN-422 |
| 5-Androstenediol (3beta,17beta-dihydroxy-androst-5 | |
| ene) | |
| 5-Androstenedione (androst-5-en-3,17-dione) | |
| 5-Methoxy-3,4-methylenedioxyamphetamine | MMDA |
| 5-Methoxy-N,N-diisopropyltryptamine | 5-MeO-DIPT |
| Acetorphine | |
| Acetyl-alpha-methylfentanyl | |
| Acetyldihydrocodeine | Acetylcodone |
| Acetylmethadol | Methadyl acetate |
| Alfentanil | Alfenta |
| Allylprodine | |
| Alphacetylmethadol except levo-alphacetylmethadol | |
| Alpha-ethyltryptamine | ET, Trip |
| Alphameprodine | |
| Alphamethadol | |
| Alpha-methylfentanyl | China White, fentanyl |
| Alpha-methylthiofentanyl | China White, fentanyl |
| Alpha-methyltryptamine | AMT |
| Alphaprodine | Nisentil |
| Alprazolam | Xanax |
| Aminorex | has been sold as methamphetamine |
| Amobarbital | Amytal, Tuinal |
| Amobarbital & noncontrolled active ingred. | |
| Amobarbital suppository dosage form | |
| Amphetamine | Dexedrine, Adderall, Obetrol |
| Anabolic steroids | “Body Building” drugs |
| Androstanedione (5alpha-androstan-3,17-dione) | |
| Anileridine | Leritine |
| Aprobarbital | Alurate |
| Barbital | Veronal, Plexonal, barbitone |
| Barbituric acid derivative | Barbiturates not specifically listed |
| Benzethidine | |
| Benzoylecgonine | Cocaine metabolite |
| Benzphetamine | Didrex, Inapetyl |
| Benzylmorphine | |
| Betacetylmethadol | |
| Beta-hydroxy-3-methylfentanyl | China White, fentanyl |
| Beta-hydroxyfentanyl | China White, fentanyl |
| Betameprodine | |
| Betamethadol | |
| Betaprodine | |
| Bezitramide | Burgodin |
| Bolasterone (7alpha,17alpha-dimethyl-17beta- | |
| hydroxyandrost-4-en-3-one) | |
| Boldenone (17beta-hydroxyandrost-1,4-diene-3-one) | Equipoise, Parenabol, Vebonol, dehydrotestosterone |
| Bromazepam | Lexotan, Lexatin, Lexotanil |
| Bufotenine | Mappine, N,N-dimethylserotonin |
| Buprenorphine | Buprenex, Temgesic, Subutex, Suboxone |
| Butabarbital (secbutabarbital) | Butisol, Butibel |
| Butalbital | Fiorinal, Butalbital with aspirin |
| Butobarbital (butethal) | Soneryl (UK) |
| Butorphanol | Stadol, Stadol NS, Torbugesic, Torbutrol |
| Calusterone (7beta,17alpha-dimethyl-17beta- | Methosarb |
| hydroxyandrost-4-en-3-one) | |
| Camazepam | Albego, Limpidon, Paxor |
| Carfentanil | Wildnil |
| Cathine | Constituent of “Khat” plant, (+)-norpseudoephedrine |
| Cathinone | Constituent of “Khat” plant |
| Chloral betaine | Beta Chlor |
| Chloral hydrate | Noctec |
| Chlordiazepoxide | Librium, Libritabs, Limbitrol, SK-Lygen |
| Chlorhexadol | Mechloral, Mecoral, Medodorm, Chloralodol |
| Chlorphentermine | Pre-Sate, Lucofen, Apsedon, Desopimon |
| Clobazam | Urbadan, Urbanyl |
| Clonazepam | Klonopin, Clonopin |
| Clonitazene | |
| Clorazepate | Tranxene |
| Clortermine | Voranil |
| Clostebol (4-chloro-17beta-hydroxyandrost-4-en-3- | Alfa-Trofodermin, Clostene, 4-chlorotestosterone |
| one) | |
| Clonazepam | Trecalmo, Rize, Clozan, Veratran |
| Cloxazolam | Akton, Lubalix, Olcadil, Sepazon |
| Coca Leaves | |
| Cocaine | Methyl benzoylecgonine, Crack |
| Codeine | Morphine methyl ester, methyl morphine |
| Codeine & isoquinoline alkaloid 90 mg/du | Codeine with papaverine or noscapine |
| Codeine combination product 90 mg/du | Empirin, Fiorinal, Tylenol, ASA or APAP w/codeine |
| Codeine methylbromide | |
| Codeine preparations - 200 mg/100 ml or 100 gm | Cosanyl, Robitussin A-C, Cheracol, Cerose, Pediacof |
| Codeine-N-oxide | |
| Cyprenorphine | |
| Dehydrochloromethyltestosterone (4-chloro-17beta- | Oral-Turinabol |
| hydroxy-17alpha-methylandrost-1,4-dien-3-one) | |
| Delorazepam | |
| Delta1-dihydrotestosterone (17beta-hydroxy-5alpha- | 1-Testosterone |
| androst-1-en-3-one) | |
| Desomorphine | |
| Dexfenfluramine | Redux |
| Dextromoramide | Palfium, Jetrium, Narcolo |
| Dextropropoxyphene dosage forms | Darvon, propoxyphene, Darvocet, Propacet |
| Dextropropoxyphene, bulk (non-dosage forms) | Propoxyphene |
| Diampromide | |
| Diazepam | Valium, Diastat |
| Dichloralphenazone | Midrin, dichloralantipyrine |
| Diethylpropion | Tenuate, Tepanil |
| Diethylthiambutene | |
| Diethyltryptamine | DET |
| Difenoxin | Lyspafen |
| Difenoxin 1 mg/25 ug AtSO4/du | Motofen |
| Difenoxin preparations - 0.5 mg/25 ug AtSO4/du | Motofen |
| Dihydrocodeine | Didrate, Parzone |
| Dihydrocodeine combination product 90 mg/du | Synalgos-DC, Compal |
| Dihydrocodeine preparations 10 mg/100 ml or 100 gm | Cophene-S, various others |
| Dihydroetorphine | DHE |
| Dihydromorphine | |
| Dimenoxadol | |
| Dimepheptanol | |
| Dimethylthiambutene | |
| Dimethyltryptamine | DMT |
| Dioxaphetyl butyrate | |
| Diphenoxylate | |
| Diphenoxylate preparations 2.5 mg/25 ug AtSO4 | Lomotil, Logen |
| Dipipanone | Dipipan, phenylpiperone HCl, Diconal, Wellconal |
| Diprenorphine | M50-50 |
| Dronabinol in sesame oil in soft gelatin capsule | Marinol, synthetic THC in sesame oil/soft gelatin |
| Drostanolone (17beta-hydroxy-2alpha-methyl- | Drolban, Masterid, Permastril |
| 5alpha-androstan-3-one) | |
| Drotebanol | Metebanyl, oxymethebanol |
| Ecgonine | Cocaine precursor, in Coca leaves |
| Estazolam | ProSom, Domnamid, Eurodin, Nuctalon |
| Ethchlorvynol | Placidyl |
| Ethinamate | Valmid, Valamin |
| Ethyl loflazepate | |
| Ethylestrenol (17alpha-ethyl-17beta-hydroxyestr-4- | Maxibolin, Orabolin, Durabolin-O, Duraboral |
| ene) | |
| Ethylmethylthiambutene | |
| Ethylmorphine | Dionin |
| Ethylmorphine combination product 15 mg/du | |
| Ethylmorphine preparations 100 mg/100 ml or 100 gm | |
| Etonitazene | |
| Etorphine (except HCl) | |
| Etorphine HCl | M 99 |
| Etoxeridine | |
| Fencamfamin | Reactivan |
| Fenethylline | Captagon, amfetyline, ethyltheophylline |
| | amphetamine |
| Fenfluramine | Pondimin, Ponderal |
| Fenproporex | Gacilin, Solvolip |
| Fentanyl | Duragesic, Oralet, Actiq, Sublimaze, Innovar |
| Fludiazepam | |
| Flunitrazepam | Rohypnol, Narcozep, Darkene, Roipnol |
| Fluoxymesterone (9-fluoro-17alpha-methyl- | Anadroid-F, Halotestin, Ora-Testryl |
| 11beta,17beta-dihydroxyandrost-4-en-3-one) | |
| Flurazepam | Dalmane |
| Formebolone (2-formyl-17alpha-methyl- | Esiclene, Hubernol |
| 11alpha,17beta-dihydroxyandrost-1,4-dien-3-one) | |
| Furazabol (17alpha-methyl-17beta- | Frazalon, Miotolon, Qu Zhi Shu |
| hydroxyandrostano[2,3-c]-furazan) | |
| Furethidine | |
| Gamma Hydroxybutyric Acid | GHB, gamma hydroxybutyrate, sodium oxybate |
| Gamma Hydroxybutyric Acid preparations | Zyrem |
| Glutethimide | Doriden, Dorimide |
| Halazepam | Paxipam |
| Haloxazolam | |
| Heroin | Diacetylmorphine, diamorphine |
| Hydrocodone | dihydrocodeinone |
| Hydrocodone & isoquinoline alkaloid <15 mg/du | Dihydrocodeinone + papaverine or noscapine |
| Hydrocodone combination product <15 mg/du | Lorcet, Lortab, Vicodin, Vicoprofen, Tussionex, Norco |
| Hydromorphinol | |
| Hydromorphone | Dilaudid, dihydromorphinone |
| Hydroxypethidine | |
| Ibogaine | Constituent of “Tabernanthe iboga” plant |
| Isomethadone | Isoamidone |
| Ketamine | Ketaset, Ketalar, Special K, K |
| Ketazolam | Anxon, Loftran, Solatran, Contamex |
| Ketobemidone | Cliradon |
| Levo-alphacetylmethadol | LAAM, long acting methadone, levomethadyl |
| | acetate |
| Levomethorphan | |
| Levomoramide | |
| Levophenacylmorphan | |
| Levorphanol | Levo-Dromoran |
| Loprazolam | |
| Lorazepam | Ativan |
| Lormetazepam | Noctamid |
| Lysergic acid | LSD precursor |
| Lysergic acid amide | LSD precursor |
| Lysergic acid diethylamide | LSD, lysergide |
| Marihuana | Cannabis, marijuana |
| Mazindol | Sanorex, Mazanor |
| Mebutamate | Capla |
| Mecloqualone | Nubarene |
| Medazepam | Nobrium |
| Mefenorex | Anorexic, Amexate, Doracil, Pondinil |
| Meperidine | Demerol, Mepergan, pethidine |
| Meperidine intermediate-A | Meperidine precursor |
| Meperidine intermediate-B | Meperidine precursor |
| Meperidine intermediate-C | Meperidine precursor |
| Meprobamate | Miltown, Equanil, Micrainin, Equagesic, Meprospan |
| Mescaline | Constituent of “Peyote” cacti |
| Mestanolone (17alpha-methyl-17beta-hydroxy- | Assimil, Ermalone, Methybol, Tantarone |
| 5alpha-androstan-3-one) | |
| Mesterolone (1alpha-methyl-17beta-hydroxy-5alpha- | Androviron, Proviron, Testiwop |
| androstan-3-one) | |
| Metazocine | |
| Methadone | Dolophine, Methadose, Amidone |
| Methadone intermediate | Methadone precursor |
| Methamphetamine | Desoxyn, D-desoxyephedrine, ICE, Crank, Speed |
| Methandienone (17alpha-methyl-17beta- | Dianabol, Metabolina, Nerobol, Perbolin |
| hydroxyandrost-1,4-diene-3-one) | |
| Methandriol (17alpha-methyl-3beta,17beta- | Sinesex, Stenediol, Troformone |
| dihydroxyandrost-5-ene) | |
| Methaqualone | Quaalude, Parest, Somnafac, Opitimil, Mandrax |
| Methcathinone | N-Methylcathinone, “cat” |
| Methenolone (1-methyl-17beta-hydroxy-5alpha- | Primobolan, Primobolan Depot, Primobolan S |
| androst-1-en-3-one) | |
| Methohexital | Brevital |
| Methyldesorphine | |
| Methyldienolone (17alpha-methyl-17beta- | |
| hydroxyestr-4,9(10)-dien-3-one) | |
| Methyldihydromorphine | |
| Methylphenidate | Concerta, Ritalin, Methylin |
| Methylphenobarbital (mephobarbital) | Mebaral, mephobarbital |
| Methyltestosterone (17alpha-methyl-17beta- | Android, Oreton, Testred, Virilon |
| hydroxyandrost-4-en-3-one) | |
| Methyltrienolone (17alpha-methyl-17beta- | Metribolone |
| hydroxyestr-4,9,11-trien-3-one) | |
| Methyprylon | Noludar |
| Metopon | |
| Mibolerone (7alpha,17alpha-dimethyl-17beta- | Cheque, Matenon |
| hydroxyestr-4-en-3-one) | |
| Midazolam | Versed |
| Modafinil | Provigil |
| Moramide-intermediate | |
| Morpheridine | |
| Morphine | MS Contin, Roxanol, Oramorph, RMS, MSIR |
| Morphine combination product/50 mg/100 ml or gm | |
| Morphine methylbromide | |
| Morphine methylsulfonate | |
| Morphine-N-oxide | |
| Myrophine | |
| N,N-Dimethylamphetamine | |
| Nabilone | Cesamet |
| Nalorphine | Nalline |
| Nandrolone (17beta-hydroxyestr-4-en-3-one) | Deca-Durabolin, Durabolin, Durabolin-50 |
| N-Benzylpiperazine | BZP, 1-benzylpiperazine |
| N-Ethyl-1-phenylcyclohexylamine | PCE |
| N-Ethyl-3-piperidyl benzilate | JB 323 |
| N-Ethylamphetamine | NEA |
| N-Hydroxy-3,4-methylenedioxyamphetamine | N-hydroxy MDA |
| Nicocodeine | |
| Nicomorphine | Vilan |
| Nimetazepam | Erimin |
| Nitrazepam | Mogadon |
| N-Methyl-3-piperidyl benzilate | JB 336 |
| Noracymethadol | |
| Norbolethone (13beta,17alpha-diethyl-17beta- | Genabol |
| hydroxygon-4-en-3-one) | |
| Norclostebol (4-chloro-17beta-hydroxyestr-4-en-3-one | Anabol-4-19, Lentabol |
| Nordiazepam | Nordazepam, Demadar, Madar |
| Norethandrolone (17alpha-ethyl-17beta-hydroxyestr- | Nilevar, Pronabol, Solevar |
| 4-en-3-one) | |
| Norlevorphanol | |
| Normethadone | Phenyldimazone |
| Normethandrolone (17alpha-methyl-17beta- | Lutenin, Matronal, Orgasteron |
| hydroxyestr-4-en-3-one) | |
| Normorphine | |
| Norpipanone | |
| Opium combination product 25 mg/du | Paregoric, other combination products |
| Opium extracts | |
| Opium fluid extract | |
| Opium poppy | Papaver somniferum |
| Opium preparations - 100 mg/100 ml or /100 gm | Parepectolin, Kapectolin PG, Kaolin Pectin P.G. |
| Opium tincture | Laudanum |
| Opium, granulated | Granulated opium |
| Opium, powdered | Powdered opium |
| Opium, raw | Raw opium, gum opium |
| Oxandrolone (17alpha-methyl-17beta-hydroxy-2-oxa- | Anavar, Lonavar, Oxandrin, Provitar, Vasorome |
| 5alpha-androstan-3-one) | |
| Oxazepam | Serax, Serenid-D |
| Oxazolam | Serenal, Convertal |
| Oxycodone | OxyContin, Percocet, Endocet, Roxicodone, Roxicet, |
| Oxymesterone (17alpha-methyl-4,17beta- | Anamidol, Balnimax, Oranabol, Oranabol 10 |
| dihydroxyandrost-4-en-3-one) | |
| Oxymetholone (17alpha-methyl-2- | Anadrol-50, Adroyd, Anapolon, Anasteron, |
| hydroxymethylene-17beta-hydroxy-5alpha- | |
| androstan-3-one) | |
| Pardroyd | |
| Oxymorphone | Numorphan |
| Para-Fluorofentanyl | China White, fentanyl |
| Parahexyl | Synhexyl, |
| Paraldehyde | Paral |
| Pemoline | Cylert |
| Pentazocine | Talwin, Talwin NX, Talacen, Talwin Compound |
| Pentobarbital | Nembutal |
| Pentobarbital & noncontrolled active ingred. | FP-3 |
| Pentobarbital suppository dosage form | WANS |
| Petrichloral | Pentaerythritol chloral, Periclor |
| Peyote | Cactus which contains mescaline |
| Phenadoxone | |
| Phenampromide | |
| Phenazocine | Narphen, Prinadol |
| Phencyclidine | PCP, Sernylan |
| Phendimetrazine | Plegine, Prelu-2, Bontril, Melfiat, Statobex |
| Phenmetrazine | Preludin |
| Phenobarbital | Luminal, Donnatal, Bellergal-S |
| Phenomorphan | |
| Phenoperidine | Operidine, Lealgin |
| Phentermine | Ionamin, Fastin, Adipex-P, Obe-Nix, Zantryl |
| Phenylacetone | P2P, phenyl-2-propanone, benzyl methyl ketone |
| Pholcodine | Copholco, Adaphol, Codisol, Lantuss, Pholcolin |
| Piminodine | |
| Pinazepam | Domar |
| Pipradrol | Detaril, Stimolag Fortis |
| Piritramide | Piridolan |
| Poppy Straw | Opium poppy capsules, poppy heads |
| Poppy Straw Concentrate | Concentrate of Poppy Straw, CPS |
| Prazepam | Centrax |
| Pregabalin | Lyrica |
| Proheptazine | |
| Properidine | |
| Propiram | Algeril |
| Psilocybin | Constituent of “Magic mushrooms” |
| Psilocyn | Psilocin, constituent of “Magic mushrooms” |
| Pyrovalerone | Centroton, Thymergix |
| Quazepam | Doral |
| Racemethorphan | |
| Racemoramide | |
| Racemorphan | Dromoran |
| Remifentanil | Ultiva |
| Secobarbital | Seconal, Tuinal |
| Secobarbital & noncontrolled active ingred | |
| Secobarbital suppository dosage form | |
| Sibutramine | Meridia |
| SPA | 1-dimethylamino-1,2-diphenylethane, Lefetamine |
| Stanozolol (17alpha-methyl-17beta-hydroxy-5alpha- | Winstrol, Winstrol-V |
| androst-1-eno[3,2-c]-pyrazole) | |
| Stenbolone (17beta-hydroxy-2-methyl-5alpha- | |
| androst-1-en-3-one) | |
| Stimulant compounds previously excepted | Mediatric |
| Sufentanil | Sufenta |
| Sulfondiethylmethane | |
| Sulfonethylmethane | |
| Sulfonmethane | |
| Talbutal | Lotusate |
| Temazepam | Restoril |
| Testolactone (13-hydroxy-3-oxo-13,17-secoandrosta- | Teolit, Teslac |
| 1,4-dien-17-oic acid lactone) | |
| Testosterone (17beta-hydroxyandrost-4-en-3-one) | Android-T, Androlan, Depotest, Delatestryl |
| Tetrahydrocannabinols | THC, Delta-8 THC, Delta-9 THC and others |
| Tetrahydrogestrinone (13beta,17alpha-diethyl-17beta- | THG |
| hydroxygon-4,9,11-trien-3-one) | |
| Tetrazepam | Myolastan, Musaril |
| Thebacon | Acetylhydrocodone, Acedicon, Thebacetyl |
| Thebaine | Precursor of many narcotics |
| Thiamylal | Surital |
| Thiofentanyl | Chine white, fentanyl |
| Thiopental | Pentothal |
| Tiletamine & Zolazepam Combination Product | Telazol |
| Tilidine | Tilidate, Valoron, Kitadol, Lak, Tilsa |
| Trenbolone (17beta-hydroxyestr-4,9,11-trien-3-one) | Finaplix-S, Finajet, Parabolan |
| Triazolam | Halcion |
| Trimeperidine | Promedolum |
| Vinbarbital | Delvinal, vinbarbitone |
| Zaleplon | Sonata |
| Zolpidem | Ambien, Ivadal, Stilnoct, Stilnox |
| Zopiclone | Lunesta |

Ethylenediamines
mepyramine (pyrilamine)
antazoline

Ethanolamines
diphenhydramine
carbinozamine
doxylamine
clemastine
dimenhydrinate

Alkylamines
pheniramine
chlorphenamine (chlorpheniramine)
dexchlorphenamine
brompheniramine
triprolidine

Piperazines
cyclizine
chlorcyclizine
hydroxyzine
meclizine

Tricyclies
promethazine
alimemazine (trimeprazine)
cyproheptadine
azatadine
ketotifen

Second-generation H₁-receptor antagonists
acrivastine
astemizole
cetirizine
loratadine
mizolastine
terfenadine (withdrawn from most markets due to risk of cardiac arrhythmias and replaced with fexofenadine)

Third-generation H₁-receptor antagonists
levocetirizine
desloratadine
fexofenadine

Other agents

Inhibitors of histamine release
cromoglicate (cromolyn)
nedocromil

H₃-receptors antagonists
Thioperamide
Clobenpropit
Impromidine

H₄-receptors antagonists
Thioperamide

Antihistamines
Aminoalkyl ethers
Bromazine, Carbinoxamine, Clemastine, Chlorphenoxamine, Diphenylpyraline, Diphenhydramine, Doxylamine
Substituted alkylamines
Brompheniramine, Chlorphenamine, Dexbrompheniramine, Dexchlorpheniramine, Dimetindene, Pheniramine, Talastine
Substituted ethylene diamines
Chloropyramine, Histapyrrodine, Mepyramine, Methapyrilene
Phenothiazine derivatives
Alimemazine, Hydroxyethylpromethazine, Isothipendyl, Mequitazine, Methdilazine, Oxomemazine, Promethazine
Piperazine derivatives Buelizine, Cetirizine, Chlorcyclizine, Cinnarizine, Cyclizine, Levocetirizine, Meclozine, Oxatomide
Others for systemic use
Acrivastine, Antazoline, Astemizole, Azatadine, Azelastine, Bamipine, Cyproheptadine, Deptropine, Desloratadine, Ebastine, Epinastine, Ketotifen, Loratadine, Mebhydrolin, Mizolastine, Phenindamine, Pimethixene, Pyrrobutamine, Rupatadine, Terfenadine, Triprolidine
Antiallergic agents excluding corticosteroids
Antazoline, Azelastine, Fexofenadine
Other antiallergies
Emedastine, Epinastine, Ketotifen, Olopatadine

STATINS
Atorvastatin, Cerivastatin, Fluvastatin, Lovastatin, Mevastatin, Pitavastatin, Pravastatin, Rosuvastatin, Simvastatin

ACE inhibitors
Captopril Enalapril Lisinopril Perindopril Ramipril Quinapril Benazepril Cilazapril Fosinopril Trandolapril Spirapril Delapril Moexipril Temocapril Zoefenopril Imidapril

Angiotensin II antagonists
Losartan Eprosartan Valsartan Irbesartan Candesartan Telmisartan Olmesartan medoxomil diuretics

Barbiturates
Allobarbital, Amobarbital, Aprobarbital, Barbexaclone, Barbital, Butabarbital, Butalbital, Butobarbital, Cyclobarbital, Ethallobarbital, Heptabarbital, Hexobarbital, Mephobarbital, Metharbital, Methohexital, Methylphenobarbital, Pentobarbital, Phoeobarbital, Primidone, Proxibarbal, Reposal, Secobarbital, Talbutal, Thiobarbital, Thiopental, Vinbarbital, Vinylbital

BETA BLOCKERS
Historical
Dichloroisoprenaline Practolol Pronethaolol
Non-selective agents
Alprenolol Carteolol Levobunolol Mepindolol Metipranolol Nadolol Oxprenolol Penbutolol Pindolol Propranolol Sotalol Timolol
β₁-Selective agents
Acebutolol Antenolol Betaxolol Bisoprolol Esmolol Metoprolol Nebivolol
Mixed α₁/β-adrenergic antagonists
Carvedilol Celiprolol Labetalol
β₂-Selective agents

Butoxamine

Benzodiazepines
Adinazolam, Alprazolam, Bromazopam, Brotizolam, Camazepam, Chlordiazepoxide, Cinolazepam, Clobazam, Clonazepam, Clorazepate, Clotiazepam, Diazepam, Doxefazepam, Estazolam, Ethyl loflazepate, Etizolam, Fluidazepam, Flunitrazepam, Flurazepam, Gidazepam, Halazepam, Ketazolam, Loprazolam, Lorazepam, Lormetazepam, Medazepam, Midazolam, Nitrazepam, Nordazepam, Oxazepam, Pinazepam, Prazepam, Quazepam, Temazepam, Tetrazepam, Triazolam, Ciglitazone, englitazone

Others:
Acarbose, miglitot, voglibose
Repaglinide, gliquidone, nateglinide
Gliclazide, glimepiride
Aliskiren, remikiren
Zolasartin, cilexitil, tasosartan
Fentiapril, pivalopril, zofenopril, alacepril, amalopril, spirapril, indolapril, pentopril, cilazapril, imidapril
Dronedarone

In addition, it would be understood that these listed active drugs can exist as different optical isomers, as different polymorphs, or as salts, derivatives, prodrugs or metabolites of the named active ingredient. It would be understood that the invention encompasses each of these variations of the named active drug, and is intended to be included as part of the claimed invention.

The term “undetectable amount” means that when using conventional analytical techniques such as high performance liquid chromatography (HPLC), nuclear magnetic resonance imaging (NMRI), and the like, the presence of an active compound can not be identified. The term “pharmacologically ineffective amount” means an amount of a drug or drugs that has or have no measurable pharmacological effect. Due to the conditions under which high speed automated tabletting equipment are operated, mixing of different granulations may occur during tablet formation which may cause material such as drug substance present in one granulation to appear in a layer or segment where it was not intended to be placed.

The term “relatively inactive segment” refers to a segment that either contains an undetectable amount of any drug or contains a decreased concentration of any drug or drugs contained in another segment or segments in a pharmacologically effective quantity. The term “decreased concentration” means that the concentration of a drug or drugs in said relatively inactive segment is no more than 80% that of said drug or drugs in another segment, more preferably no more than 20% of said other segment'3 s drug or drugs concentration; most preferably said ratio is no more than 5%, however. The concentration of a drug or drugs in a segment means, herein, the ratio, on a weight to weight basis, of the drug or drugs in said segment to the total weight of said segment, which includes said drug or drugs and inactive excipients.

The tablets of the invention are preferably broken transversely in order to realize their benefits or advantages. They may be broken in standard ways, according to the invention such as either by applying force manually (or “by hand” as the term is commonly understood) to cause the tablet to break at a desired location, or by use of an instrument, such as a cutting edge, to apply force directly to a separation mark provided in a desired breaking region.

Separation marks are intended to guide optional tablet breaking in the usual manner that is well known with scores, so that, if tablet breaking is desired, force can be applied to break the tablet at or about the separation mark in a direction that is substantially perpendicular to the surface on which it is desired that breakage of the tablet will be initiated. The tablet according to the invention may be broken either by applying force manually or by an instrument such as a cutting edge directly to the separation mark, or to other areas of the tablet, such as the outer segments, to cause the tablet to break at or about the separation mark and in the direction of the separation mark.

The separation mark or marks may comprise one or more of the following:
(a) a score in a side wherein said score is not oriented vertically;
(b) indicia on at least one side or lateral face of the tablet that indicates or locates a desired breaking region of said tablet;
(c) a band which is located on one segment or at an interface of two segments; or
(d) an inner segment of said tablet in which a first lower and a second upper segment have the same color and contain either the same drug in a pharmacologically effective quantity or both lack a pharmacologically effective quantity of any drug, and the third, inner or interposed segment that has a different color from said first segment and has either the same drug as said first segment when said first segment has a pharmacologically effective quantity of a drug or has no pharmacologically effective quantity of a drug when said first segment lacks a pharmacologically effective quantity of any drug.

### EXAMPLES

The subject invention can be readily understood by describing specific examples, which are intended as illustrative of the invention, and are not intended as limiting.

1 SPECIFIC DRUGS
a. Warfarin

As one example, warfarin sodium may be usefully produced as a trisected, quadrisected, or pentasected tablet, which can be accurately broken into predictable partial doses. If the tablet were taken whole, such a scoring pattern would be irrelevant. Preferably, the quadrisected warfarin tablet can be provided as a tablet which is manufactured according to the techniques, and to provide the advantages of the tablets disclosed in WO 2005/112900 and WO 2006/038916. A trisected product may also be produced according to known techniques with a five layer tablet such as one that can be produced with the Korsch TRP 900, in which, for example, the first, third, and fifth layers (segments) comprise equal and therapeutic amounts of warfin sodium, and the second and fourth layers (segments) lack significant amounts of warfarin sodium.

Methods of treatment of the invention involve breaking a warfarin tablet into three tablets or four tablettes followed by ingestion of the appropriate dose. Most preferably this method involves breaking a trisected or quadrisected tablet that readily can be broken into predictable doses such as thirds of the whole tablet or quarters of the whole tablet. Breaking a warfarin tablet into four parts has been taught against.
b. Torsemide

Another example involves the antihypertensive agent torsemide, which has favorable properties at low doses. A trisected 6 mg tablet would be novel and useful, especially if accurately divisible into three 2 mg tablettes. Also, a useful and advantageous composition considered part of the subject invention is a quadrisected 8 mg torsemide tablet, breakable into four 2 mg “tablettes. ”A quadrisected 4 mg torsemide tablet would be useful to produce the known effective antihypertensive doses of 2, 3, and 4 mg doses; and the 1 mg tablet can be useful as a low dose alternative to provide antihypertensive efficacy in cases of an adverse reaction to a higher dose or in pediatric cases, and may have activity against hypertension in some patients. Torsemide tablets according to the subject invention may be provided as scored tablets embodied as shown in FIGS. 1, 2, or 3, above, wherein the tablet comprises torsemide as the active ingredient rather than warfarin.

As with numerous other embodiments of the invention, it has not been taught to treat hypertension with torsemide by beginning at a fractional part of a whole tablet, whether that is a half dose, third dose, or quarter dose, then increasing the dose such as to a whole tablet from a half tablet, a half tablet from a quarter tablet, etc. based on clinical results, side effects, and the like. “Dose adjustment” includes without limitation upward and downward dose adjustments, which occur often on beginning a new medication, ending a medication, or during ongoing therapy with a medication. “Dose adjustment” also includes periodic dosing, such as is common with a hypnotic agent or a tranquilizer, where doses vary based for example on level of sleepiness (hypnotic), level of anxiety (tranquilizer), or need to drive a car (benzodiazepine, etc.). The method of the invention as applied to torsemide and the many other drugs where such method of dose adjustment has not been taught may involve scored or unscored drugs, and predictably breaking or unpredictably breaking drugs with regard to the doses created by tablet breaking. Of course, it is most preferred under the invention that these methods involve predictable dosing upon tablet splitting.
c. Lamotigine

In another example, the anticonvulsant lamotrigine is currently produced as a bisected tablet including a 100 mg dose, but is instructed to be taken whole. It may therefore be advantageous to produce, for example, a quadrisected 100 mg lamotrigine tablet if predictable 25 mg dosages (tablettes) can readily be obtained by quartering said tablet according to the quadrisect pattern. Lamotrigine tablets according to the subject invention may be provided as scored tablets embodied as shown in FIGS. 1, 2, or 3, above, wherein the tablet comprises lamotrigine as the active ingredient rather than warfarin. Dose titration could be as provided in the PI for Lamictal®, but using fractional tablets rather than the whole tablets specified in the PI.
d. Glyburide

Glyburide is currently typically provided as scored 1.25, 2.5 and 5 mg tablets. Dosing is up to 20 mg daily in either once or twice daily doses. In the PI for drug products containing glyburide as the active ingredient, dose titration is mentioned but no instruction to split the scored tablets is expressly recited or otherwise provided. In accordance with the subject invention, the use of a scored 2.5 mg tablet which is predictably and accurately divisible into two 1.25 mg tablettes can be employed to achieve a daily dose of 3.75 mg without having to purchase a 1.25 mg tablet in addition to the 2.5 mg tablet. In accordance with the subject invention, a predictably and accurately breakable 5 or 10 mg quadrisected tablet may also be utilized. Dosing in this example begins using ¼ tablet daily and may increase to the use of half tablets or a combination of a quarter-, half- or whole tablet as tolerated and as glycemic response necessitates. Additionally, the initial tablet utilized could be a 4, 6 or 8 mg tablet. There is no requirement in the subject invention that introduction of a new version and/or a new method of administration requires dosing to utilize the same doses as were previously available.

Less preferred dosing methods of the invention, again, can involve tablets provided with one or more separation marks that break relatively unpredictably, such as by patients, or by trained pharmacists.
c. L-Thyoxine

Synthroid® (L-thyroxine) is currently marketed in the U.S. as a scored tablet (apparently a decorative score). Again, despite the presence of a score, no PI instruction exists to utilize the drug as a half-tablet. Dosing per the invention may, for example, utilize a 100 mcg quadrisected tablet, with instructions to the patient to begin using ¼ tablet (predictably providing a tablette containing an accurate 25 mcg dose) daily for 8 days (a total of two 100 mcg tablets), then ½ tablet daily (predictably providing a tablette containing an accurate 50 mcg dose) for 24 days (i.e., administering a total of six 100 mcg tablets), then to have an office visit and blood test to determine the appropriateness of a further dose increase to 75 or 100 mcg. Thus, a single prescription and two visits to the physician are required by this dosing schedule using a dosage form which is breakable into predictably accurate doses. By contrast, previous dosing schedules would require at least three prescriptions (one each for 25, 50, and 75 or 100 mcg tablets, and often a physician visit for each new prescription. Therefore, prescription costs and medical expenses such as physician visitation costs can be substantially decreased by employing compositions and methods in accordance with the subject invention.

Prominent brands of this thyroid replacement hormone are Synthroid® and Levoxyl®. Each has a bisect mark or marks that is (are) shallow and likely non-functional. The PI's for each product do not mention tablet splitting. Levoxyl is shaped like a thyroid gland. The titration schemes in the PI's involve what could beneficially be used as half or quarter doses of whole tablets. The Synthroid.com Web site discusses money-saving actions consumers can take, including buying a larger quantity of drug at one time and using a coupon. Not mentioned as a way to save money is tablet splitting, even though pricing rises much less proportionately than dosage.

The invention teaches a non-homogeneous pharmaceutical tablet comprising L-thyroxine, preferably a tablet in which L-thyroxine is the only drug. The invention may involve a taller-than-wide tablet with one or more non-L-thyroxine-containing and preferably drug-free segments, and two or more segments comprising L-thyroxine.

This may for example involve a tablet of the structure AIA, AIAIA, AIAIAIA. The “I” segments are optionally and preferably all scored.

In another preferred embodiment, the invention involves a compressed tablet comprising unitary segments in which L-thyroxine is preferably the only drug. The scoring pattern may most preferably involve bisection, trisection, or quadrisection; or, less preferably, pentasection through “octa-section” (e.g., seven parallel scores in an elongated tablet delineating 8 preferably equal sections of the tablet). Less preferred are tablets in which L-thyroxine is contained within deeply scored rather than unitary segments.

It is believed that L-thyroxine has not been taught to be scored beyond bisection, and in fact the manufacturers in the U.S. of L-thyroxine do not recommend breaking said tablets through the score, and do not price their tablets as if they were intended to broken or were to be utilized as broken.

Thus the invention also involves L-thyroxine-containing pharmaceutical tablets with more than a bisect score pattern.

The invention also involves novel methods of dose titration and dose adjustment. The Synthroid and Levoxyl labels contain similar methods of initiating L-thyroxine for different types of patients and for different conditions. Advantageously, the novel method utilizes the inventions to allow dose titration and dose adjustment to be performed conveniently by a patient without necessarily having to purchase or obtain an additional strength. For example, a patient beginning treatment on 25 mcg of L-thyroxine daily could in the instant invention's method utilize a quadrisected 100 mcg L-thyoxine tablet, a quarter of a tablet at a time. Dose increments could be to a half a tablet and then if needed to a whole tablet.

Utilization of an accurately breakable dosage form would enhance the utility of the invention. Dose adjustment by breaking a tablet other than the lowest manufactured dose of 25 mcg is also novel if it involves breaking in half a tablet of a higher L-thyroxine strength. It will be apparent that scoring patterns of trisection, quadrisection, and even more complex scoring patterns allow more complex dosage titration/adjustment regiments than bisection.
f. Olanqapine and anti-psychotic agents

Olanzapine is marketed in the US as Zyprexa®. This “atypical” anti-psychotic agent is indicated for Tx of mania as well as psychosis.

Dosing involves titration, often starting with 5 mg. Zyprexa/olanzapine tablets are unscored in the US and bisected or unscored elsewhere.

Dose-related side effects, such as somnolence and hypotension, are present with Zyprexa and tend to affect the elderly and the frail patients the most.

Novel tablets containing olanzapine as, preferably, the only drug include those with a plurality of unitary segments comprising olanzapine; a deeply scored segment comprising olanzapine; and taller than wide tablets comprising a plurality of segments comprising olanzapine, preferably separated by a substantially inactive segment(s).

Specifically claimed are tablets comprising olanzapine with a trisect, quadrisect, or more complex than quadrisect scoring pattern. These tablets of the instant invention are preferably but not necessarily accurately and readily breakable.

Novel methods of treatment involve dosage titration and dosage adjustment using an olanzapine-containing tablet, ideally but not necessarily one that is accurately breakable with regard to dose. The greatest flexibility will be obtained from a tablet that is trisected or quadrisected rather than one that is bisected, though the invention involves treating with bisected tablets for the purpose of dose adjustment and titration.

Olanzapine is also provided in combination with fluoxetine (Symbyax®) in the US. Similar methods and pharmaceutical tablets for this combination, and similar methods for other potential combinations, as described herein for olanzapine as the only drug in the tablet, are part of the invention.

Several other antipyschotic medications have been developed. These include quetiapine, risperidone, paliperidone, ziprasidone, and Seroquel®. Whether in immediate release (“IR”) or controlled release (“CR”) form, the methods and novel scored trisected or quadrisected tablets as applicable to olanzapine, and any combinations containing these medications, are part of the invention.

Chemically distinct but pharmacologically containing similar therapeutic effect and similar adverse dose-related effects involving somnolence and hypotension are antipsychotic phenothiazines. Examples include haloperidol, Compazine®, promethazine and many others.
g. Alpha-beta blocking agents as well as alpha blocking and beta blocking agents

These agents have a variety of therapeutic uses. Beta blockers are typically alcohols. Certain beta blocking agents also have peripheral effects on alpha adrenergic receptors, blocking these receptors and are therefore called alpha-beta blocking agents. These drugs are used for treatment (“Tx”) of HTN, CHF, arrhythmia, migraine prophylaxis, and other uses. Separately, relatively pure alpha blocking agents are used for hypertension and prostatic dysfunction.

Novel scored tablets, and novel methods of use, are disclosed herein for members of these drug classes.

I. Alpha-beta blockers.
Examples: Carvedilol, labetalol Carvedilol (Coreg®) has been utilized in immediate release fashion for years for Tx of hypertension (“HTN”) and congestive heart failure (“CHF”). This product is known to have been produced as unscored and bisected tablets. For CHF it is always taught to be titrated upwards from subtherapeutic low doses, such as 3.125 mg BID. A new product has been developed, Coreg CR®, a CR version comprising carvedilol. Also produced is a combination of carvedilol and lisinopril.

The invention involves novel scored tablets comprising carvedilol as the only drug, or carvedilol and lisinopril as the only drugs. These scoring patterns involve trisected, quadrisected, and more complex than quadrisected tablets. Preferably these tablets are readily breakable into fractional predictable doses.

Methods of use involve dose adjustment and dose titration. Examples include the follow.

Carvedilol for CHF.

Provide a starting dose of 3.125 mg BID as ½ of 6.25 mg tablet given BID. Most preferably this involves an accurately breakable dosage form. In addition, this starting dosage could involve a preferably accurately breaking quadrisected 12.5 mg carvedilol tablet, which could then also be utilized to provide 6.25 mg BID and then 12.5 mg BID. Ongoing dosage adjustments could also be provided by such tablets. Doses involving bisected or quadrisected 25 mg or 50 mg tablets could be used, as well. Dosing for hypertension does not typically utilize the 3.125 mg dose, but BID usage would provide utility for the invention in such ways as using a quadrisected 25 mg carvedilol to allow a starting dose of 6.25 mg BID, then upward titration to 12.5 mg Bid, followed by 25 mg Bid or even a simplified regimen of 25 or 12.5 mg QD.

Recently, Coreg CR, a capsule formulation of immediate and controlled release microparticles containing carvedilol, has been approved for marketing in the U.S. The label emphasizes the importance of careful up-dose titration, beginning with 10 mg QD. The invention can involve placing the components of Coreg CR into tablet form, with appropriate scoring or tablet format as needed to allow 20 mg or 40 mg, for example, to be used as partial tablets to provide a 10 mg starting dose for dose initiation for CHF. Extensive dosage adjustments and titration dosage variations are expected to be needed based on individual sensitivities, presence of diuretic treatment, and medical conditions, for example. As Coreg CR has beta-blocking capabilities, downward titration with care is expected to be needed in some patients so that issues of rapid beta-blocket withdrawal will not be unnecessarily raised. Accurate dosage adjustments will provide important medical benefits for this product, especially when it is used for CHF treatment.

Alpha Blockers

Terazosin, doxazosin, prazosin and tamsulosin for benign prostatic hyperplasia (or, benign prostatic hypertrophy, “BPH” in either terminology) and all of the above other than tamsulosin

I. Benign prostatic hypertrophy (“BPH” or benign prostatic hyperplasia)

Because of a first-dose effect that may cause syncope, the antihypertensive drugs terazosin, doxazosin, and prazosin in their immediate release forms require 1 mg starting doses and then upward titration to a therapeutic dose.

The invention involves utilizing these drugs in up-titration schemes and during ongoing therapy by starting with a fractional tablet to provide the 1 mg starting dose and then either a whole 2 mg tablet or such as a fractional 4, 6 or even 8 mg tablet to provide an increased, 2 mg dose, and then increasing doses as needed. Even though these drugs are often provided once daily, they can also be used as BID drugs. This dose regimen also can very usefully involve a fractional tablet taken BID rather than two smaller strength tablets each taken QD. Because of the side effects, accurate dosing is very important for this class of drug, as it is for chemically unrelated drugs such as hydralazine and other direct vasodilators. Alpha blockers and alpha-beta blockers are among the drugs for which the invention teaches a method of dose titration on dose initiation which, while novel and having certain potential benefits, it would be clearly medically preferred to have dosing be accurate upon tablet splitting. It would not be obvious to quadrisect an alpha-blocker or alpha-beta blocker unless accurate dosing upon tablet breaking could occur. Under current thinking and knowledge, it would currently not be obvious to place a decorative score into an alpha blocker tablet, especially a trisect or quadrisect pattern, because patients would tend to misuse the score to guide tablet splitting rather than accepting the score as decorative only. This issue of misuse of decorative scores is relevant to many other embodiments of the invention, where the prior art involves decorative scores only into a tablet, whether they are bisect or even quadrisect decorative scores. The invention's preferred tablets overcome this problem by providing highly functional scores as well as unscored tablets that break into tablettes containing predictable doses. Similar uses for the above three drugs relate to their use for BPH as well as HTN.

In the case of tamsulosin, the drug can be produced as a tablet of 0.4 bisected, 0.6 trisected, and 0.8 mg trisected to allow a variety of dosing adjustments. This could include beginning 0.4 mg therapy with a half of 0.8 mg and then up-titrating if indicated, otherwise continuing on ½ tablet daily. Due to orthostatic hypotension, the more accurate the tablet splitting, the better medically it would be. A quadrisected 0.8 mg tablet and a trisected 0.6 mg tablet would allow significant dosage flexibility. For example, certain men would benefit from 0.4 mg HS and 0.2 mg in the AM, which is beneficially able to be provided by a 0.6 mg trisected tablet if accurate dosing can be obtained by breaking said tablet in said manner. Of course, similar benefits in safety and/or efficacy could accrue to a quadrisected 0.8 mg tamsulosin tablet. Less preferred would be a single-scored 0.6 mg tamsulosin tablet which divided the tablet into 0.4 and 0.2 mg rather than a bisecting score. As is the case for all the other tablets described herein, unscored but accurately dosed tablets can be created by using a taller than wide tablet with one or more interposed non-tamsulosin-containing (often, preferably inactive) segments.

In the case of beta blockers that lack alpha-blocking ability, such as metotprolol, atenolol, and many others, the ability to titrate or dose adjust by using preferably accurately breakable dosage forms can be very beneficial. A patient could for example begin therapy with atenolol 25 mg BID by breaking a quadrisected 100 mg atenolol tablet, then increase the dose as needed to 50 mg BID or even to 75 mg in the AM and 25 mg in the PM. A trisected 150 (or 75) mg atenolol could be beneficial if utilized to start therapy at 50 (25) mg once daily and then increase the dosing in various ways as allowed by the scoring pattern (or breaking pattern for dosage forms involving interposed preferably inactive segments between active drug-containing segments of appropriate therapeutic or at least pharmaceutically active dosage.

2. TREATMENT METHODS
a. Hypercholesterolemia

In the treatment of hypercholesterolemia, current methods employed in the U.S. include beginning with the maximum dose that is hoped by the prescriber to be tolerated, taking into account such factors as a patient's age, weight, hepatic function, cholesterol level, and/or current and desired cholesterol levels. Because potential side effects such as myalgias and rhabdomyolysis are dose dependent, physicians tend to start with a lower “safe” dose, even if that dose is predicted to be inadequate to bring a patient to a desired goal. Because all statins marketed in the U.S. are currently unscored, are likely to be difficult to break and therefore provide unpredictable doses if broken or otherwise divided, physicians often initially prescribe a lower dose than they expect to ultimately be needed. Thus atorvastatin may be prescribed at 10 mg daily, then, as shown to be tolerated by the patient, new prescriptions may be subsequently given for 20 mg and then 40 mg tablets. This dosing routine may require multiple visits to a physician or pharmacy, additional costs, and other disadvantageous aspects.

In accordance with the subject invention, dosing may begin with one-quarter of a 40 mg tablet, which predictably provides an accurate 10 mg dose. Then, when tolerability of this 10 mg dose is demonstrated, such as after 4 or 8 days, dosing may increase to one-half of the 40 mg tablet daily (providing a predictable 20 mg dose), and potentially then to one 40 mg tablet daily. Advantageously, using the compositions and methods of the subject invention, it is expected that patient compliance with the ultimate goal of dosing 40 mg daily will be increased, as compared to purchasing three separate tablet strengths, for reasons that include the starting dose appearing cautiously low at only ¼ of a tablet daily. Moreover, the compositions and methods of the subject invention are advantageous in that fewer visits to the physician or receipt of prescriptions from the physician for any one patient are required. A preferred means of utilizing the invention as in this example of a quadrisected statin tablet is to utilize the inventions described in International Appliction, WO 2005/112900.

It is also expected that the method of the invention will increase the percentage of physicians who get patients to goal. It has been well documented that many physicians under-treat generally asymptomatic conditions such as elevated hyperlipidemia, so that having a high dose that can be initially used as a low dose is likely to increase the number of physicians who utilize that dose. Not only do compositions and methods of the subject invention allow titration upwards to occur in a fully acceptable manner, but if a patient receives a dosage that is double the starting dose, an accurately breakable dosage form allows the patient to go to, for example, a half dose (i.e., said starting dose) if a side effect appears at the higher dose that was not present at the starting dose.

Another advantage of the invention applied to statins and other drugs is that it is known to the inventor that certain patients need to start as low as 5 mg daily on atorvastatin, increase the dose gradually as guided by muscle aches, but eventually “learn” to tolerate maximal doses. The invention greatly cases the ability of patients to adjust to statin doses while maintaining “full speed ahead” on dose increases as may be medically required. Similar considerations can apply to hypertension remedies, antipsychotic agents, and other drug classes.
b. HYPERTENSION

In the treatment of hypertension, whether essential or secondary, it is recognized that many patients are sensitive to medication commonly prescribed to treat this condition. It is also recognized that many patients require higher doses than the starting doses, even if their degree of hypertension is (at least initially) considered by the physician to be mild (Stage1). Thus, it is common to begin with a starting dose of a medication below that expected to be needed as a final or maintenance dose. In the invention, a predictable starting dose that is a fraction of a larger dose is utilized for an adequate period of time to demonstrate tolerability and safety, and to evaluate adequacy of dosing, then dosing is increased by providing either the whole tablet or a larger fraction of the dose thereof. Beginning treatment of hypertension with lisinopril 2.5 mg, as one-quarter of a quadrisected 10 mg dose is an example of the invention, with increasing dosing to 5 mg (one-half of a scored 10 mg dose), and then to 7.5 or 10 mg, using tablettes that are preferably predictable in dose, and/or whole tablets as needed.

Another example involves dosing schedules using an alpha-adrenergic blocking agent, such as doxazosin, terazosin, or prazosin. The mandated starting dose for each is 1 mg, preferably taken before bed. The above alpha-adrenergic blocking agent drugs are indicated for hypertension and also for benign prostatic hypertrophy/hyperplasia (“BPH”). In accordance with the method of the subject invention, an accurately breakable bisected 2 mg tablet (accurately yielding two 1 mg tablettes), trisected 3 mg tablet (accurately yielding three 1 mg tablettes, of quadrisected 4 mg tablet (accurately yielding four 1 mg tablettes) may be used to provide the 1 mg starting dose, and then upward dose adjustment would be made using the tablet of the invention, and tablettes created therefrom, as medically appropriate.
c. ANXIETY

Management of anxiety and pain also can benefit from the procedures of the invention. A physician can devise, and a PI can authorize or instruct, a treatment regimen that for example could comprise 0.5 mg (one tablet) of alprazolam nightly and 0.25 mg (=½ tablet) as needed during the day. It has not been taught to adjust doses with precision. The invention for anxiety is primarily directed to, in addition to novel scored tablets, novel methods of treatment with tablets that readily break into tablettes of predictable dose.
d. ALLERGY

In yet a different embodiment of the invention, a drug such as prednisone is commonly utilized to treat a condition such as an acute allergic or asthmatic reaction. In one of many potential examples of the usefulness of the invention, a 40 mg quadrisected prednisone tablet may come to be created which is predictably breakable into tablettes containing accurate 20 mg (halved) or 10 mg (quartered) doses, and this 40 mg tablet can be prescribed for asthma. The subject method comprises a treatment whereby the patient is initially administered a whole 40 mg tablet, then per physician's instruction, the dose may be reduced to 30 mg (¾ of a predictably and accurately breakable 40 mg tablet), or 20 mg (one-half of a predictably and accurately breakable 40 mg tablet), then administering 10 mg (¼ of a predictably and accurately breakable 40 mg tablet). The treatment method can then include administration of a dose as directed by the physician. After the patient reaches the 10 mg dose (¼ tablet), the invention may be utilized again by prescribing a sub-10 mg bisected, trisected, or quadrisected dose and having the patient taper off the medication by utilizing progressively smaller fractions of the whole dose.

In different embodiment of this de-escalation dosing regimen in accordance with the invention, an acute allergic reaction involving laryngeal edema may be treated using two 40 mg tablets, each quadrisected, then lowering the dose by 10-20 mg per day as directed utilizing whole tablets that predictably and accurately breakable into tablettes containing a fraction of the dose in the whole tablet, or fractions thereof.

In the above examples, quadrisection and dosing using a defined portion of a whole tablet is provided as a preferred embodiment of the invention. No limitation is intended. For example, a tablet may be sectioned to provide more than four sections. These and other embodiments of the invention that are contemplated by or suggested to persons of ordinary skill in the art using this disclosure and/or which is known within the art is within the scope of the invention. Alternatively, fewer than four sections of a tablet may be formed. For example, when bisection (providing two halves) is adequate to allow dose titration and adjustment, then such is also within the scope of the invention.

Further, it is preferred that accurate division of a tablet with regard to the fractional doses be obtained, such as, but not limited to the dosage forms described in published International patent applications WO 2005/112898 and WO 2005/112900 and other means of creating pharmaceutical tablets optimized for accurate breaking using known, homogeneous tablets, including elongated tablets.

Among the benefits of the subject invention are improvements in patient compliance. Patients typically prefer taking a fraction of a dose to start treatment and then increasing the dose by taking a larger portion of the tablet and/or the whole tablet. Patient compliance can be enhanced especially for asymptomatic conditions such as hypertension and hypercholesterolemia. Patient compliance is also expected to be enhanced by a decrease in the number of prescriptions required.

From a physician's standpoint, or that of other prescribers, the level of complexity of prescribing may advantageously be decreased by the methods of the invention. By use of compositions or methods of the subject invention, a prescriber can recommend safe usage of a partial dose, such as in a titration dosing scheme without concern of inaccurate doses resulting from breaking of a tablet where the inaccurate dose may not protect the patient, or may cause unwanted side effects, such as can happen in a dose-dependent way in epilepsy, diabetes, hypertension, and the like.

More generally, the prescriber can authorize a patient to achieve a predictable dose outside a titration dosing scheme. For example, a patient whose use of the drug lamotrigine (currently available only in 100 and 25 mg doses), could involve administration of 100 mg in the morning and 50 mg (one-half tablet) in the evening or before bed. Use of the currently available dosage forms would, per the label (Patient Instruction), may require two separate prescriptions, prescribed as one 100 mg tablet and a separately prescribed 25 mg tablet (two tablets for the 50 mg dose) each day. Per the invention, the patient could safely utilize one whole and one-half 100 mg tablet daily. Such use can therefore be advantageous for physicians and patients.

A further benefit of the invention may relate to pediatric or geriatric doses, which may not be produced in appropriate dose strengths. In the case of amlodipine, a 1.25 mg daily dose may be useful in either small children with hypertension, or in frail elderly patients with angina or hypertension, who may have hepatic dysfunction. Even though the United States Food and Drug Administration (FDA) has not approved a 1.25 mg dose, precise divisibility of the approved 2.5 mg dose would allow a 1.25 mg daily dose. In addition, precise divisibility of the approved 2.5 mg dose will allow accurate dosing of 3.75 mg daily.

Another use of the invention is to enable a method of cost savings to insurers and patients. The invention allows this because many drugs have pricing that differs little (if at all) between different doses. Because tablet splitting is imprecise for most scored tablets, the practice of mandatory splitting has been met with disapproval by most physician and pharmacist organizations. The invention enables tablet splitting due to provide accurate dosing when a tablet (or some tablettes) of the invention are broken as described herein. Substantial benefits are foreseen from this innovation. In addition, the ability to separate one active drug from another in a combination product has cost saving advantages, as well.

The subject invention applies to methods and compositions useful for treatment of animals, preferably mammals, and more preferably to humans.

It would be readily understood that the compositions useful for application to the methods in accordance with the subject invention may be scored or unscored. It would be further understood that the term “bisected” dosage form or tablet refers to the dosage from bearing one or more marks or scores indicating divisibility of that dosage form into two portions or fractional doses, preferably two equal portions or halves. The bisecting mark or score may be a single line, for example a score line collinear with the diameter of a conventional round tablet, or may be transverse to the longest dimension of a capsule-shaped tablet. Similarly, references herein to a “trisected” dosage form or tablet identifies a dosage form bearing one or more marks or scores that indicate division of the dosage form into three portions, preferably three equal portions or thirds. “Quadrisected,” accordingly, refers to at least one mark or score in or on a dosage form that indicates division of the dosage form into four portions, preferably four equal portions or quarters. Further numbers of divisions of a dosage form into five or more portions or fractional doses may also be employed in the subject invention, and may only be limited by the result of requiring a predictably accurate lower dose upon such division of the dosage form.

Moreover, the subject invention further includes an article of manufacture, or kit, that comprises a finished dosage form which is breakable or otherwise divisible into a predictably accurate lower dose, and an instruction, preferably a written or electronic instruction for breaking the dosage form and administering a fractional dose of said finished dosage form, or divided dosage form. Preferably, the article of manufacture includes a packaged tablet or tablets and a separate instruction for use in accordance with the subject method, more preferably co-packaged for sale as a single unit.

It is recognized that related inventions may be within the spirit of the disclosures herein. Also no omission in the current application is intended to limit the inventors to the current claims or disclosures. While certain preferred and alternative embodiments of the invention have been set forth for purposes of disclosing the invention, modifications to the disclosed embodiments may occur to those who are skilled in the art.

Other drug products, e.g., drug products comprising any of the active ingredients described or incorporated herein, can be embodied in tablets uniquely scored with the subject score patterns to advantageously provide predictably accurate lower or partial doses to a patient. Accordingly, the examples and the accompanying drawings are not intended to be limiting, and are provided for illustration purposes only.

1. An improved pharmaceutically acceptable tablet containing at least one drug wherein said at least one drug is provided in a prior art dosage form as a capsule, an unscored tablet, a tablet having a bisecting score pattern, a tablet having a trisecting score pattern, or a tablet having a quadrisecting score pattern, said improvement comprising:
a tablet when the prior art dosage form is not a tablet;
a tablet having a score pattern selected from the group consisting of a bisecting, trisecting, quadrisecting, a pentasecting, and a score pattern more complex than a pentasecting score pattern, when the prior art tablet is limited to an unscored tablet;
a tablet having a score pattern selected from the group consisting of a trisecting, quadrisecting, a pentasecting, and score pattern more complex than a pentasecting score pattern when the prior art tablet is limited to an unscored tablet or a bisected tablet;
a tablet having a score pattern selected from the group consisting of a bisecting, quadrisecting, a pentasecting, and a score pattern more complex than a pentasecting score pattern, when the prior art dosage form is a trisected tablet; or
a tablet having a score pattern selected from the group consisting of a bisecting, trisecting, and a pentasecting score pattern when the prior art dosage form is a quadrisected tablet; wherein said score pattern is formed by a debossed score or separation mark.

2. The tablet of claim 1 comprising a scoring pattern defining five or more substantially equal regions in which a substantially equal amount of drug or combination of drugs is present in each regions defined by the score pattern.

3. The tablet of claim 2 in which said tablet is readily divisible into said five or more equal amounts of drug or drugs.

4. The tablet of claim 1 comprising an active pharmaceutical ingredient selected from the group consisting of: an anticoagulant, a statin, a non-statin lipid modifier, a cardiovascular agent, an antihypertensive agent, an angiotensin receptor antagonist, an antipsychotic agent, an atypical antipsychotic agent, a hypnotic agent, a benzodiazepine, an antidepressant, a steroid, a hypoglycemic agent, an anticonvulsant, a GABA (gamma-aminobutyric acid) receptor agonists, an antihistaminergic compound, a glitazone, a retinoid, a direct rennin inhibitor, an anti-retroviral agent, an immunosuppressant, a proton pump inhibitor, a barbiturate, and a narcotic.

5. The tablet of claim 4 in which said active pharmaceutical ingredient is the anticoagulant warfarin or a salt, isomer, polymorph, hydrate, metabolite, prodrug, or derivative thereof.

6. The tablet of claim 4 wherein said tablet comprises a lipid modifier as an active ingredient.

7. The tablet of claim 6 wherein said lipid modifier is a statin.

8. The tablet of claim 6 wherein said tablet comprises a trisecting or quadrisecting score pattern.

9. The tablet of claim 6 wherein said active pharmaceutical ingredient is ezetimibe, or a salt, isomer, polymorph, hydrate, metabolite, prodrug, or derivative thereof.

10. A method of treating, preventing or ameliorating a medical condition in a human or other animal using a dosing regimen comprising administration of different doses the human or other animal, said method comprising:
providing a whole tablet comprising a dose of one or more drugs, said tablet being breakable to provide at least two portions of said tablet such that each portion comprises a predictable fractional dose of said one or more active drugs contained in the whole tablet, said fractional dose being more predictable than a fractional dose available from a prior art dosage form manufactured using known formulation technologies and having the same drug or drugs;
prescribing or administering a fractional dose of said one or more active drugs, said fractional dose obtained by breaking the whole tablet into its one or more portions comprising a predictable fractional dose.

11. The method of claim 10 wherein said fractional dose is a predictable half, third, or quarter fractional dose of the dose contained in the whole tablet.

12. The method of claim 10 in which said accurate breaking of the tablet or a portion thereof is readily performed by a patient in a home setting.

13. The method of claim 12 in which said patient is elderly, arthritic, or blind.

14. The method of claim 10 wherein said tablet comprises a plurality of layered compositions forming the tablet.

15. The method of claim 14 wherein said layered tablet comprises a plurality of segments wherein at least one segment comprises active drug and at least one segment comprises a composition substantially free of active drug.

16. The method of claim 15 wherein said segment comprising the composition substantially free of active drug is an inner segment oriented between two end segments, each end segment comprising active drug.

17. The method of claim 10 wherein said whole tablet comprises a score or separation mark for guiding a user to break said dosage form in an area or region designated by said score or separation mark.

18. The method of claim 10 in which said medical condition is selected from the group consisting of hypertension, hyperlipidemia, diabetes mellitus, hypothyroidism, benign prostatic hyperplasia, asthma, pain, and allergy.

19. The method of claim 10 wherein said fractional dose is administered as an initial dose in a increasing dose titration regimen.

20. The method of claim 10 wherein said fractional dose is administered integral with a decreasing dose titration regimen.

21. The method of claim 10, said fractional dose being administered integral with a dosing regimen using different doses, the method providing the capability to increase or decrease the dose of the drug while retaining predictability of the altered dose.

22. The method of claim 10 wherein said dosage form comprises a drug selected from the group consisting of: an anticoagulant, a statin, a non-statin lipid modifier, a cardiovascular agent, an antihypertensive agent, an angiotensin receptor antagonist, an antipsychotic agent, an atypical antipsychotic agent, a hypnotic agent, a benzodiazepine, an antidepressant, a steroid, a hypoglycemic agent, an anticonvulsant, a GABA (gamma-aminobutyric acid) receptor agonists, an antihistaminergic compound, a glitazone, a direct renin inhibitor, an immunosuppressant, a proton pump inhibitor, a barbiturate, and a narcotic.

23. The method of claim 10 wherein said administration of the fractional dose provides a desired therapeutic or clinical end-point in treating said medical condition.

24. The method of claim 10, said method providing improved patient compliance in dose administration of a medication.

25. An article of manufacture or kit comprising:
a whole dosage form comprising a medicament, said dosage form being accurately and predictably breakable into a plurality of fractional doses of a drug or drugs, and
a separate instruction to create the fractional dose by breaking said whole dosage form or a fractional dose thereof into the plurality of fractional doses of said drug or drugs, wherein said dosage form and instruction are co-packaged as a single unit.

26. The article of manufacture or kit of claim 25 in which said instruction also includes notification that breaking the tablet as directed will produce a predictable dose of said drug or drugs.

27. The tablet of claim 1, said tablet comprising a bisecting, trisecting, or quadrisecting score pattern consisting essentially of parallel score lines or separation marks provided on one face of the tablet, said parallel lines delineating substantially equal portions of the mass of the tablet.

28. The tablet of claim 1 wherein said scoring pattern is a bisecting, trisecting, or quadrisecting score pattern consisting essentially of parallel score lines or separation marks on a single face of the tablet, said parallel lines delineating substantially equal portions of the quantity of a drug in the tablet.

29. The tablet of claim 28 where said score pattern further consists of a single score line on a different face of said tablet, said tablet comprising a drug which is ineffective for treating anxiety or depression.

30. The tablet of claim 1 wherein said tablet comprises at least five (5) segments, wherein the first, third and fifth segments each comprise a substantially identical pharmaceutically active composition, and the second and fourth segments each comprise a pharmaceutically inactive composition and said second and fourth segments comprise a separation mark.

31. The tablet of claim 30 wherein in said first, third and fifth segments comprise the same drug or drugs in substantially equal amounts.

32. The tablet of claim 1, said tablet comprising a first inactive segment adjacent to three unitary active segments formed by dividing one or more layers forming the unitary active segments.

33. The tablet of claim 1 comprising a drug marketed as a plurality of strengths selected from a single-dose strength, a double-dose strength dose, a triple-dose strength dose, and a quadruple-dose strength dose.

34. The method of claim 11 wherein breaking of the tablet is performed manually.

35. The method of claim 11 wherein said breaking of the tablet is performed with an instrument selected from a knife, a razor blade, or commercially available tablet splitter or tablet cutter that may utilize a razor blade or other cutting means.

36. The tablet of claim 1 having a bisecting separation mark or an inactive layer positioned above and below and contiguous with substantially identical active segments, wherein said tablet is readily breakable by a person to provide a plurality of tablettes each comprising a fractional dose of a drug contained in the whole tablet, wherein the fractional dose in a tablette is a predictable dose which meets the criteria selected from the group consisting of:
i) a tablette containing a partial dose of a whole tablet has substantially the same quantity of drug as a whole tablet containing the partial dose;
ii) each tablette containing a partial dose created by breaking the whole tablet as guided by a separation mark thereon contains the same amount of drug as in another tablette containing the same partial dose; and
iii) after breaking a plurality of whole tablets into its corresponding tablettes and grouped according to smallest, smaller, larger or largest sizes of resultant tablettes, each group of tablettes each comprising a fractional dose having substantially the same amount of drug as another group of tablettes,
wherein the amount of drug is determined according to a standard United States Pharmacopoeia content uniformity assay.

37. A method of producing for use in a dosing regimen comprising administration of a plurality of doses, a predictable dose of a drug or drugs contained in a whole tablet, said method comprising breaking a tablet of claim 1 into a plurality of tablettes and administering a tablette to provide a fraction of the whole tabler's dose of the drug or drugs.

38. The method of claim 37, said method comprising breaking through an inactive segment or layer of said tablet.

39. The pharmaceutical tablet of claim 1, said drug or drugs selected from the group consisting of zolpidem, doxepin, eszopiclone, carvedilol, metoprolol, L-thyroxine, digoxin, digitoxin, warfarin sodium, lamotrigine, ezetimibe, atorvastatin, metformin, pioglitazone, rosiglitazone, and zopiclone, or a salt, isomer, polymorph, hydrate, metabolite, prodrug, or derivative thereof.

40. The method of claim 21 werhein said initial treatment dose is a half dose provided as a half tablet, said method further comprising ascertaining the safety of said half dose, then increasing said dose to a whole dose contained in a whole tablet after the half dose is ascertained as safe.

41. The pharmaceutical tablet of claim 1, said tablet being unscored and readily broken into four tablettes each comprising substantially equal quantities of a drug.

42. The pharmaceutical tablet of claim 1 comprising a score of greater than about 0.8 mm in depth, measured from the face immediately adjacent to the score.

43. The pharmaceutical tablet of claim 1 having a score created from an embossing where said embossing is at least 0.8 mm in height and more preferably about at least 1.0 mm in height.

44. The method of claim 9 in which said treatment comprises administration of a lipid-lowering drug using a portion of a whole tablet.

45. The method of claim 44 in which said lipid-lowering drug is a statin.

46. The method of claim 45 in which the statin is selected from atorvastatin or rosuvastatin.

47. The method of claim 45 wherein said method facilitates a patient reaching a goal of lower blood lipid measurement, including low density lipoprotein (“LDL”) goal.

48. The method of claims 45-48 in which the tablet breaks into a fractional dose that is more predictable than a fractional dose provided by a known dosage form comprising the same drug or drugs.

49. The method of claims 45-49 in which the tablet is a segmented tablet comprising a scored drug-free segment and provides the more predictable fractional dose of the drug when broken through said score.

50. The article of manufacture of claim 25 comprising a single agent antihypertensive drug for treatment of moderate to severe hypertension, e.g., Stage 2 hypertension, said instruction providing a direction for a patient to be administered a starting dose of said antihypertensive drug product higher than recited in a prior Product Information instruction or any other authoritative document for that antihypertensive drug.

51. The article of manufacture as in claim 50 in which the instruction is directed to chronic treatment.

52. The article of manufacture as in claim 50 in which a test dose that is less than said higher starting dose is used for up to ten days to assess safety and tolerability.

53. The article of manufacture as in claim 52 in which the instruction directs a patient to subsequently lower the dose by breaking a whole tablet and administering a fractional dose from said broken whole tablet.

54. A method for treating hypertension comprising initial administration to a patient of an antihypertensive agent at a dose greater than a maximum starting dose previously recommended for said antihypertensive agent.

55. The method of claim 54 wherein the patient has a systolic blood pressure at least about 20 mm Hg above goal, or diastolic blood pressure at least about 10 mm Hg above goal (Stage 2).

56. The method of claim 54 in which the patient has hypertension but not accelerated hypertension.

57. The method of claim 56 in which the patient has Stage 2 hypertension.

58. The method of claim 54 in which the starting dose of the invention is at least 50% greater than the previously recommended maximum starting dose.

59. The method of claim 54 in which the starting dose of the invention is at least double that of the maximum previously recommended starting dose.

60. The method of claim 54 in which the starting dose of the invention is preceded by a dose within the known starting dose range for up to ten days.

61. The method of claim 54 in which the starting dose of the invention is a tablet indicated for subdivision.

62. The method of claim 15 in which said tablet is scored.

63. The method of claim 15 in which said tablet is readily breakable to produce a fractional dose that is more predictable than a fractional dose obtained by breaking a prior dosage form comprising the antihypertensive agent.

64. The method of claim 54 wherein said antihypertensive agent is selected from the group consisting of calcium antagonist (calcium channel blocker, or CCB), beta blocker, diuretic, alpha-beta blocker, angiotensin converting-enzyme inhibitor (ACE), angiotensin receptor antagonist (ARB), and direct renin inhibitor (DRI).

65. The method of claim 9 comprising utilizing a beta blocking or alpha-beta blocking drug.

66. The method of claim 65 comprising utilizing a once-daily or twice daily formulation of carvedilol or utilizing a onec-daily dosing of nebivolol.

67. The method of claim 66 in which hypertension, congestive heart failure, or post-myocardial infarction state is treated.

68. The method of claim 67 in which a predictable fractional dose of either carvedilol or nebivolol is used to treat hypertension.

69. The method of claim 68 in which patients are aided in reaching goal blood pressure safely and expeditiously by utilizing a predictable dose derived from breaking a tablet.
